(19) United States
(12) Patent Application Publication
(10) Pub. No.: US 2025/0275892 A1
(43) Pub. Date: Sep. 4, 2025

(54) COSMETIC COATING DEVICE

(71) Applicant: WOORIKIDS+, INC., Yeonsu-gu (KR)

(72) Inventors: Hoisook KIM, Incheon (KR); Jungmin LEE, Suwon-si (KR); Hyojoo KIM, Incheon (KR); Milim KIM, Incheon (KR)

(73) Assignee: WOORIKIDS+, INC., Yeonsu-gu (KR)

(21) Appl. No.: 19/093,718

(22) Filed: Mar. 28, 2025

Related U.S. Application Data
(63) Continuation of application No. PCT/KR2025/002843, filed on Feb. 28, 2025.

(30) Foreign Application Priority Data
Feb. 29, 2024 (KR) 10-2024-0030161
Feb. 29, 2024 (KR) 10-2024-0030162
Feb. 28, 2025 (KR) 10-2025-0026696
Feb. 28, 2025 (KR) 10-2025-0026697

(51) Int. Cl. A61K 8/02

(52) U.S. Cl. CPC A61K 8/0208; A61K 2800/805; A61K 2800/87

(57) ABSTRACT

Disclosed is a cosmetic coating device according to various embodiments. The cosmetic coating device may include a composition supply part applying a composition in a flowing state on one surface of a support body, a solidifying treatment part receiving the support body having the composition applied thereon from the composition supply part and performing a curing operation on the support body having the composition applied thereon, and a sheet forming part manufacturing a sheet using the support body having the one surface on which the composition is solidified after passing through the solidifying treatment part.

### CROSS-REFERENCE TO RELATED PATENT APPLICATIONS

This application is a Continuation of International Application No. PCT/KR2025/002843, filed on Feb. 28, 2025, which claims the benefit of Korean Patent Application Nos. 10-2025-0026696, filed on Feb. 28, 2025, 10-2025-0026697, filed on Feb. 28, 2025, 10-2024-0030161, filed on Feb. 29, 2025, and 10-2024-0030162, filed on Feb. 29, 2025, in the Korean Intellectual Property Office, the disclosure of which is incorporated herein in its entirety by reference.

### TECHNICAL FIELD

The present disclosure relates to a coating device for manufacturing a cleansing sheet, and more particularly, to a cosmetic coating device of which a temperature and a wavelength are adjustable that can mass-produce efficient cleansing sheets with consistent quality in rolls by applying a liquid composition at a predetermined thickness on release paper, solidifying the liquid composition through light and temperature adjustment, and allowing the liquid composition to be well adhered to the release paper.

### BACKGROUND ART

Cleansing sheets have become an indispensable item in modern life and play an essential role in keeping a user's skin clean and effectively removing daily and professional makeup. Since these cleansing sheets are disposable products, convenience and product efficiency provided to consumers are very important evaluation criteria, and the quality and performance of the product that satisfy these two factors are directly related to consumer satisfaction. To this end, a method in which a liquid composition is applied to release paper and dried to form a cleansing sheet is generally used during a manufacturing process. This process constitutes a basic manufacturing step of the product and directly affects the efficacy and usability of the final product.

However, various limitations have been found in the existing manufacturing technique. In particular, ensuring uniformity of thickness in the process of applying the liquid composition has emerged as a major challenge, and uneven application has been identified as one of the main causes of reduced product performance. Further, precise temperature and time control required in the process of solidifying the composition is difficult to adequately handle with existing equipment and technology, and as a result, the composition is not solidified at an appropriate speed and condition, which makes it difficult to maintain consistent product quality.

Furthermore, inefficiency of the process of drying and solidifying the composition not only causes a decrease in productivity but also leads to a problem in adhesion of the composition to the release paper. The problem in adhesion is a main cause of reduced quality during storage of the product or reduced performance during use and causes consumer concern about stability and reliability of the product.

### DISCLOSURE

### Technical Problem

The present disclosure is directed to providing a cleansing sheet manufacturing device for improving quality and production efficiency of a cleansing sheet.

Objectives of the present disclosure are not limited to that mentioned above, and other unmentioned objectives should be clearly understood by those of ordinary skill in the art from the description below.

### Technical Solution

A cosmetic coating device according to various embodiments of the present disclosure for addressing the above objectives is disclosed. The cosmetic coating device may include a composition supply part applying a composition in a flowing state on one surface of a support body, a solidifying treatment part curing the support body having the composition applied thereon by the composition supply part, and a sheet forming part manufacturing a sheet using the support body having the composition applied thereon.

In an alternative embodiment, the coating device may further include a support body supply part on which a support body roll having a roll shape is mounted and a controller controlling operations of the composition supply part, the solidifying treatment part, and the sheet forming part, and the support body unwound from the support body roll may sequentially pass through the composition supply part and the solidifying treatment part and may be wound in the sheet forming part.

In an alternative embodiment, the composition supply part may include a roller part including two rollers provided to form a predetermined separation distance or more, a position adjustment part adjusting a position of one of the two rollers to change a gap between the two rollers, a base part guiding input of the composition toward the roller part, and an initial curing module performing initial solidifying treatment on the applied composition when the composition is applied on the one surface of the support body.

In an alternative embodiment, the coating device may further include an input part acquiring composition characteristic information corresponding to the composition, and the controller may control an operation of at least one of the support body supply part and the sheet forming part so that the support body roll or the sheet is unwound or wound based on the composition characteristic information.

In an alternative embodiment, the controller may control an operation of the position adjustment part based on the composition characteristic information to adjust the gap between the two rollers.

In an alternative embodiment, the initial curing module may be provided above the support body to perform initial curing treatment on the composition applied on the support body and may be provided to include a plurality of light sources supplying light of different wavelength bands, and the light sources adjacent to each other may be disposed to supply light of different wavelength bands.

In an alternative embodiment, the solidifying treatment part may include a housing forming an inner space, an inlet formed in one surface of the housing, an outlet formed in each of other surfaces corresponding to the one surface, a support surface provided in the inner space and supporting the support body input through the inlet from below, a light irradiation module provided above the support surface, and a temperature adjustment part changing a temperature of the inner space.

In an alternative embodiment, the inner space may be provided to be divided into a plurality of areas to perform control in different temperature ranges and may include a first area in which a temperature is adjusted to be in a range of 25° C. to 100° C., a second area in which a temperature is adjusted to be in a range of 50° C. to 130° C., and a third area in which a temperature is adjusted to be in a range of 50° C. to 110° C.

In an alternative embodiment, the solidifying treatment part may further include a sensor part sensing environment information of the inner space and a heating part provided on an inner side of the support surface to supply heat.

In an alternative embodiment, the solidifying treatment part may include a separation membrane dividing the inner space into the plurality of areas, the separation membrane may be provided to change a size of a movement passage between adjacent areas, and the controller may control an operation of the separation membrane based on environment information of each of the plurality of areas.

Other detailed matters of the present disclosure are included in the detailed description and drawings.

### Advantageous Effects

According to various embodiments of the present disclosure, it is possible to provide a novel cleansing sheet manufacturing device that enables even application of a liquid composition, precise control of drying and solidifying processes, and maintenance of high quality and performance of the final product.

In this way, efficiency of a cleansing sheet manufacturing process can be maximized, and consumer satisfaction can be improved.

Advantageous effects of the present disclosure are not limited to those mentioned above, and other unmentioned advantageous effects should be clearly understood by those of ordinary skill in the art based on the description below.

### DESCRIPTION OF DRAWINGS

Various aspects will now be described with reference to the drawings and like reference numerals will generally be used to designate like components. In the following embodiments, for the purpose of description, multiple detailed matters are presented to provide general understanding of one or more aspects. However, it will be apparent that the aspect(s) can be executed without the detailed matters.

FIG. 1 illustrates an exemplary flowchart of a cosmetic coating method according to one embodiment of the present disclosure.

FIG. 2 illustrates an overall schematic view of a cosmetic coating device according to one embodiment of the present disclosure.

FIG. 3 illustrates an exemplary block diagram of the cosmetic coating device according to one embodiment of the present disclosure.

FIG. 4 is an exemplary view of the cosmetic coating device from one side according to one embodiment of the present disclosure.

FIG. 5 is an exemplary view of a support body supply part from one side according to one embodiment of the present disclosure.

FIG. 6 is an exemplary view of a composition supply part from a side according to one embodiment of the present disclosure.

FIGS. 7A to 7C are exemplary views of the composition supply part in various directions according to one embodiment of the present disclosure.

FIG. 8 is an exemplary view illustrating an exterior of a solidifying treatment part according to one embodiment of the present disclosure.

FIG. 9 is an exemplary view illustrating an inner side of the solidifying treatment part according to one embodiment of the present disclosure.

FIG. 10 is an exemplary view of the solidifying treatment part from a rear according to one embodiment of the present disclosure.

FIG. 11 is an exemplary view of a roll forming part from one side according to one embodiment of the present disclosure.

FIG. 12 is an exemplary view of an embossing part from one side according to one embodiment of the present disclosure.

FIGS. 13 and 14 are exemplary views for describing a bubble removing part according to one embodiment of the present disclosure.

FIG. 15 shows results according to temperature conditions.

### BEST MODE OF THE INVENTION

Various embodiments and/or aspects will now be disclosed with reference to the drawings. In the following description, for the purpose of description, multiple detailed matters will be disclosed in order to help overall understanding of one or more aspects. However, those of ordinary skill in the art of the present disclosure will recognize that the aspect(s) can be executed without the detailed matters. In the following description and the accompanying drawings, specific exemplary aspects of one or more aspects will be described in detail. However, the aspects are exemplary and some of various methods in principles of various aspects may be used and the descriptions are intended to include all of the aspects and equivalents thereof. Specifically, in an “embodiment,” “example,” “aspect,” “illustration,” and the like used in the specification, it may not be construed that a predetermined aspect or design which is described is more excellent or advantageous than other aspects or designs.

Hereinafter, the same or similar components will be assigned the same reference numbers throughout the drawings, and redundant description thereof will be omitted. Further, in describing an embodiment disclosed herein, detailed description of a related known art will be omitted if it is determined that the detailed description may obscure the gist of the embodiment disclosed herein. Further, the accompanying drawings are only for facilitating understanding of the embodiment disclosed herein, and the technical spirit disclosed herein is not limited by the accompanying drawings.

Although the terms “first,” “second,” and the like are used to describe various elements or components, these elements or components are not limited by these terms, of course. These terms are only used to distinguish one element or component from another element or component. Therefore, a first element or component to be mentioned below may be a second element or component in the technical spirit of the present disclosure.

Unless otherwise defined, all terms (including technical and scientific terms) used herein may be used with meanings that may be commonly understood by those of ordinary skill in the art to which the present disclosure pertains. Also, terms defined in commonly used dictionaries should not be interpreted in an idealized or overly formal sense unless expressly so defined herein.

In addition, the term “or” is intended to mean not exclusive “or” but inclusive “or.” That is, unless specified otherwise or clearly stated in the context, “X uses A or B” is intended to mean one of the natural inclusive substitutions. That is, “X uses A or B” may apply to any of the case where X uses A, the case where X uses B, or the case where X uses both A and B. Further, it should be understood that the term “and/or” used herein designates and includes all possible combinations of one or more items among listed related items.

Also, the terms “include” and/or “including” should be understood as indicating the presence of corresponding features and/or components but not excluding the presence or addition of one or more other features, components, and/or groups thereof. Also, unless specified otherwise or the context clearly indicates singularity, a singular expression should be generally interpreted as indicating “one or more” in the present specification and the claims.

When a certain component is mentioned as being “connected” or “linked” to another component, it should be understood that the component may be directly connected or linked to the other component, or another component may be present therebetween. On the other hand, when a certain component is mentioned as being “directly connected” or “directly linked” to another component, it should be understood that no other component is present between the two components.

The words “module” and “unit” for components used in the following description are given or used interchangeably in consideration of ease of writing the specification and do not have meanings or roles that are distinct from each other by themselves.

When a component or layer is referred to as being “on” another component or layer, this includes not only a case in which the component or layer is right above the other component or layer but also a case in which another layer or component is interposed therebetween. On the other hand, when a component is referred to as being “directly on” or “right above” another component, this indicates that no other component or layer is interposed therebetween.

Spatially relative terms such as “below,” “beneath,” “lower,” “above,” and “upper” may be used to easily describe the relationship between one component and other components illustrated in the drawings. Spatially relative terms should be understood as encompassing different directions of an element that is in use or operating, in addition to directions depicted in the drawings.

For example, if the component illustrated in the figures is turned over, a component described as “below” or “beneath” another component would then be oriented “above” the other component. Thus, the illustrative term “below” can encompass an orientation of both above and below. The components may also be oriented in different directions, and accordingly, the spatially relative terms may be interpreted according to orientations.

Objects and effects of the present disclosure and technical configurations for achieving the same will become apparent with reference to embodiments described in detail below and the accompanying drawings. In describing the present disclosure, when detailed description of known functions or configurations is determined as having the possibility of unnecessarily obscuring the gist of the present disclosure, the detailed description thereof will be omitted. Also, the terms described below are terms defined in consideration of their functionality in the present disclosure and may vary depending on an intention of a user or an operator, customary practice, or the like.

However, the present disclosure is not limited by embodiments disclosed below and may be implemented in various different forms. The present embodiments are provided to make the present disclosure complete and to fully inform those of ordinary skill in the art to which the present disclosure pertains of the scope of the disclosure, and the present disclosure is only defined by the scope of the claims. Therefore, the definition should be made based on the entire content of the present specification.

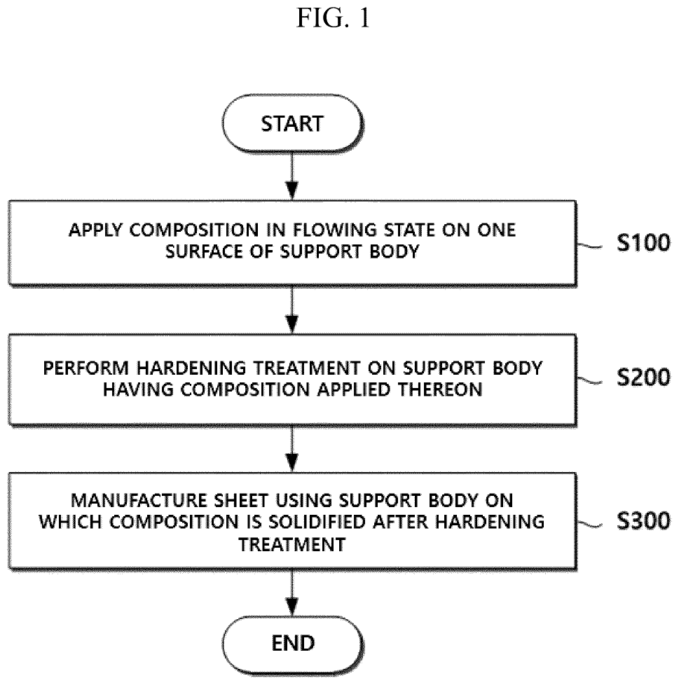

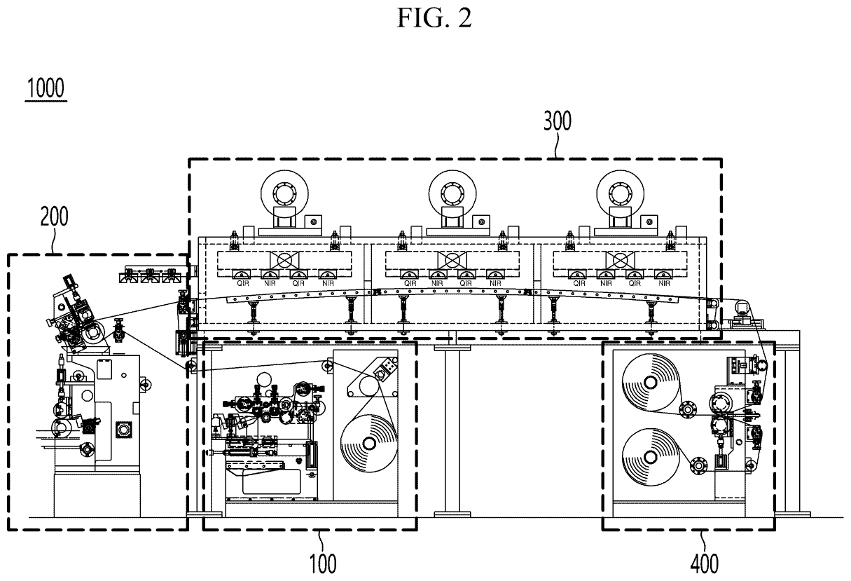

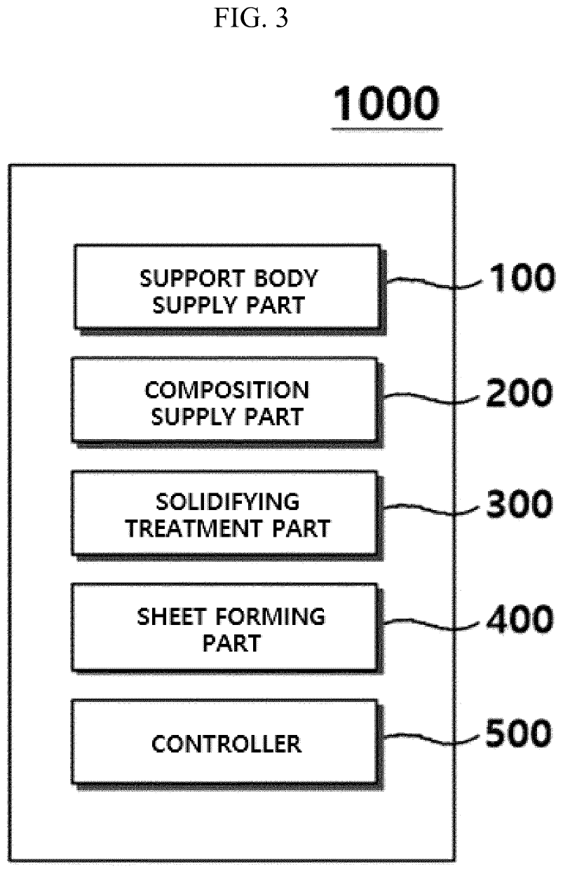

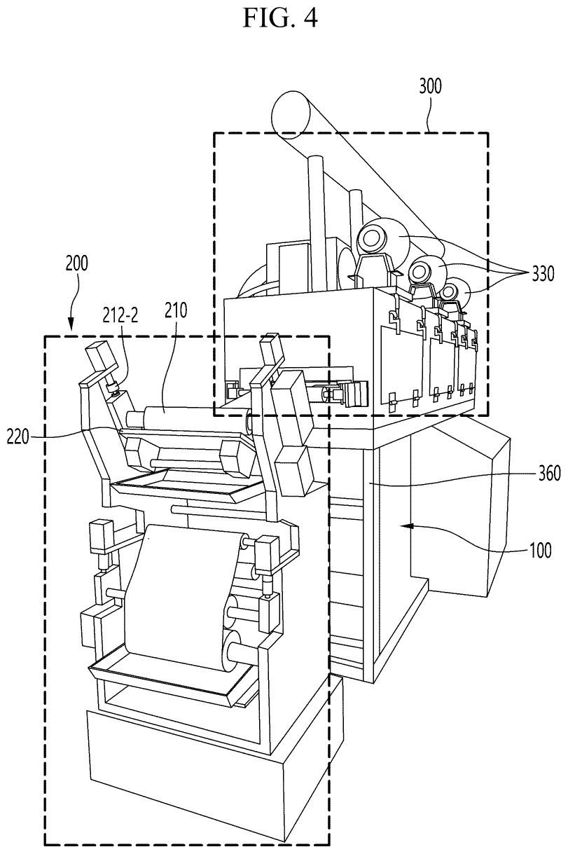

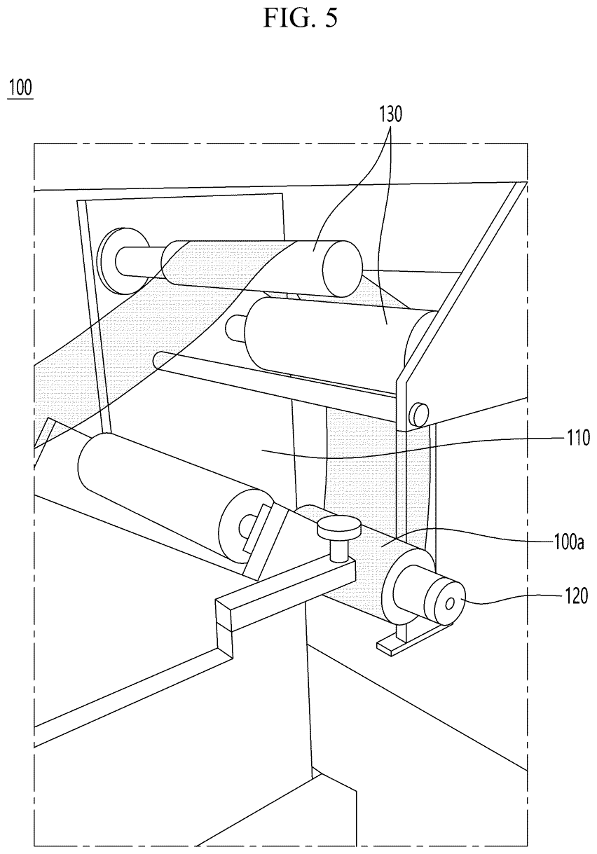

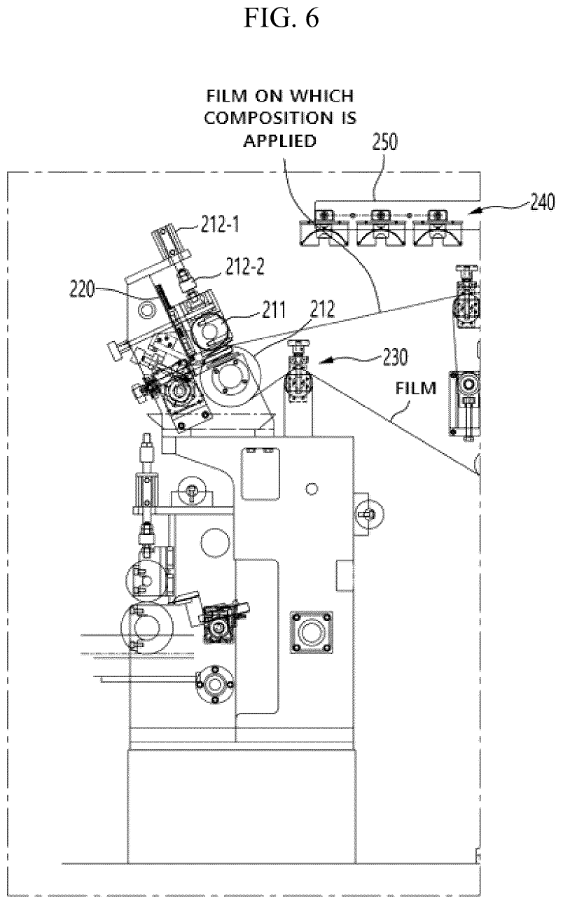

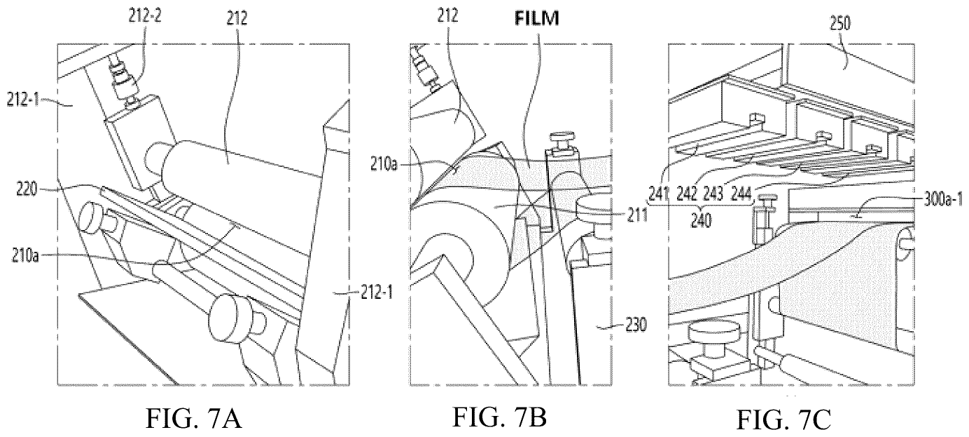

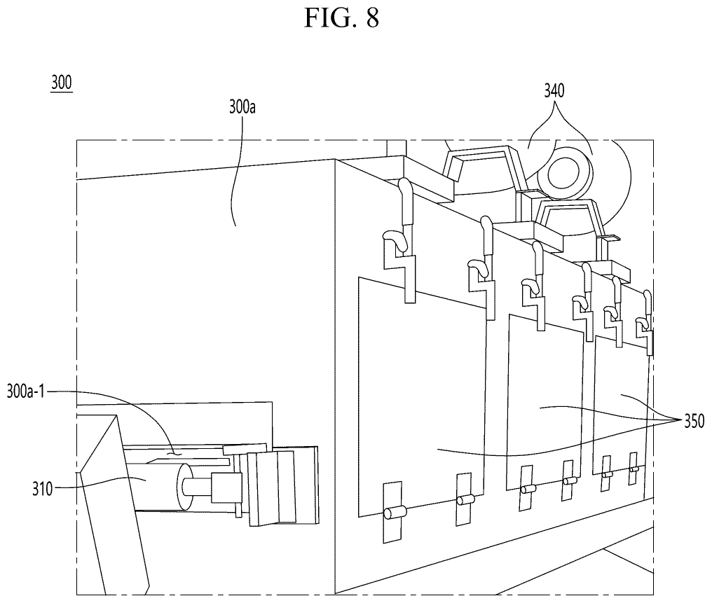

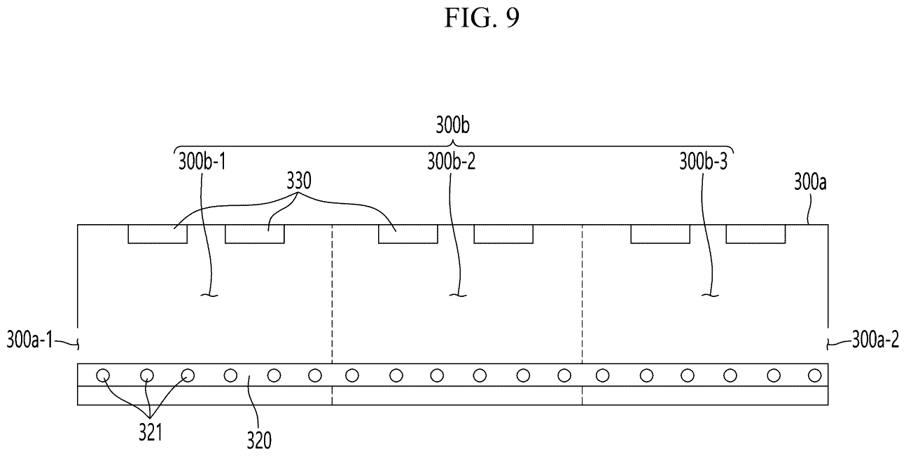

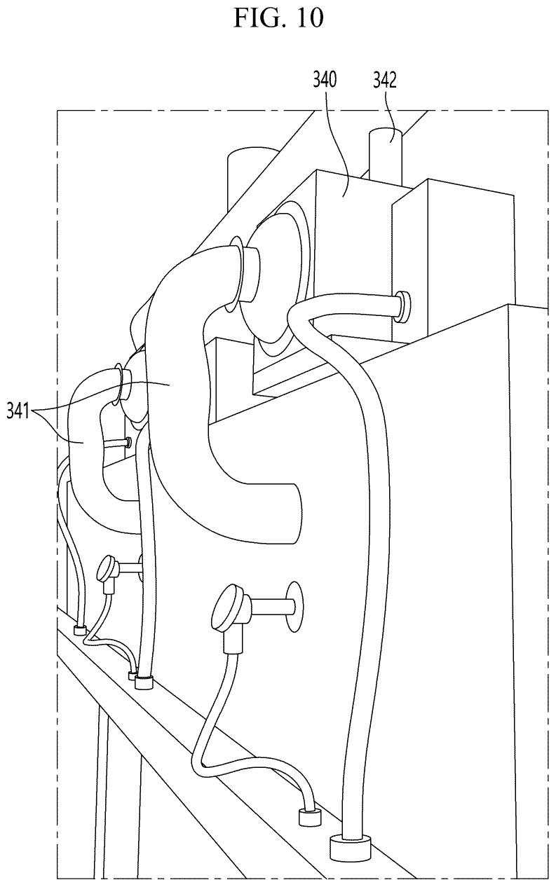

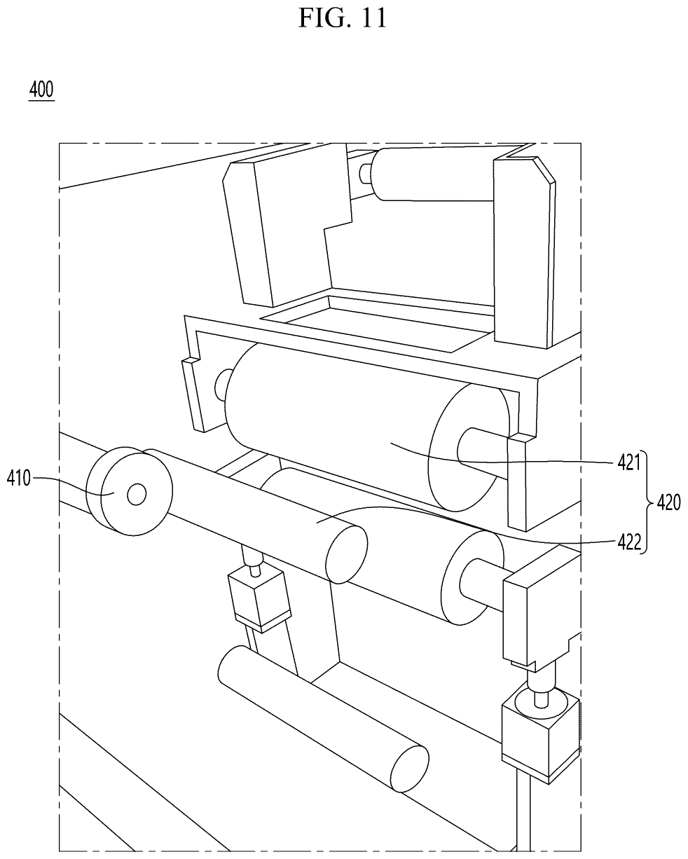

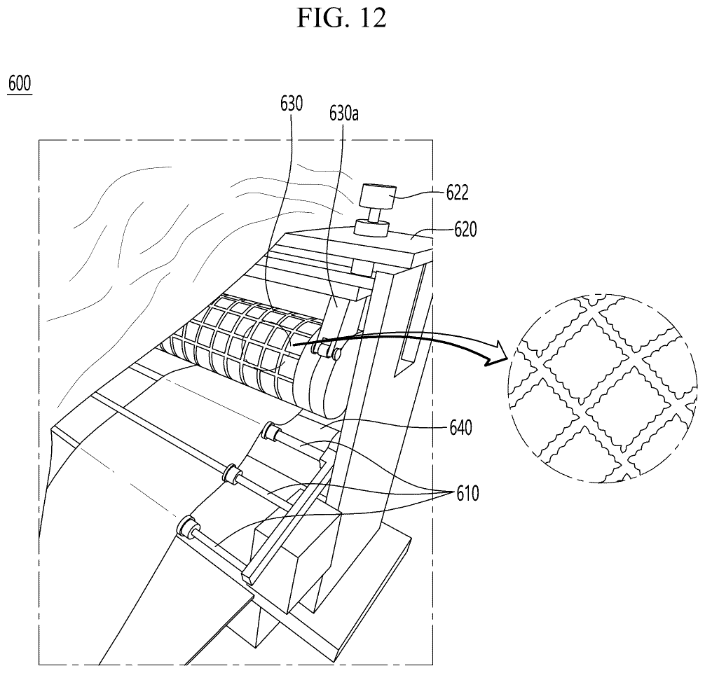

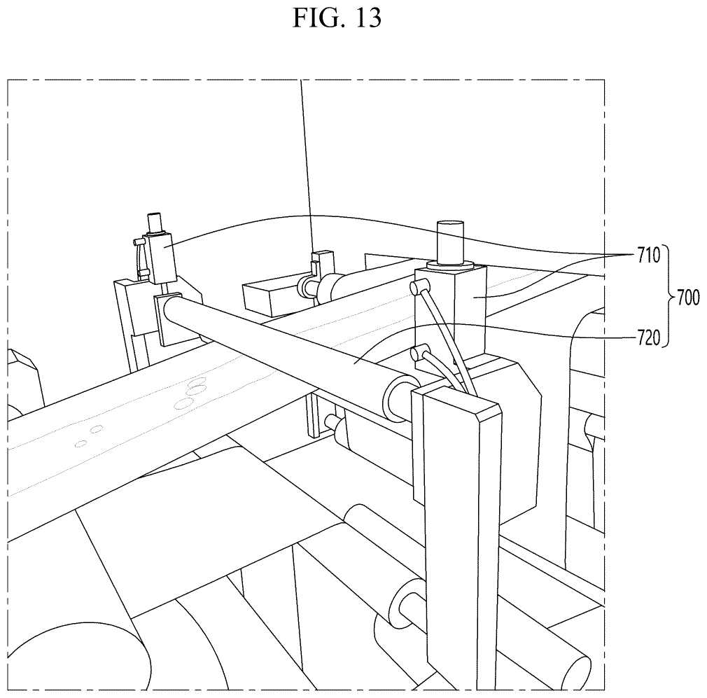

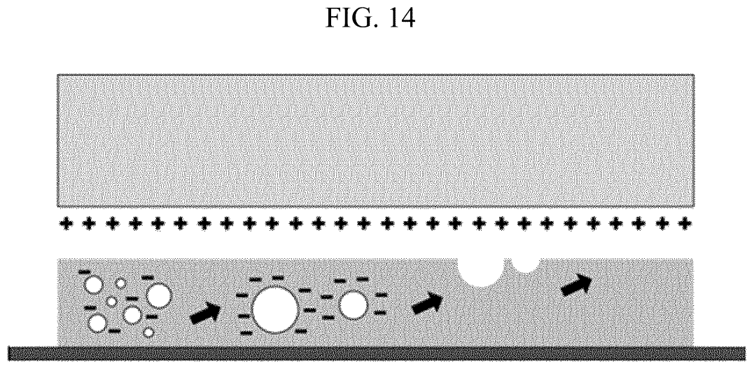

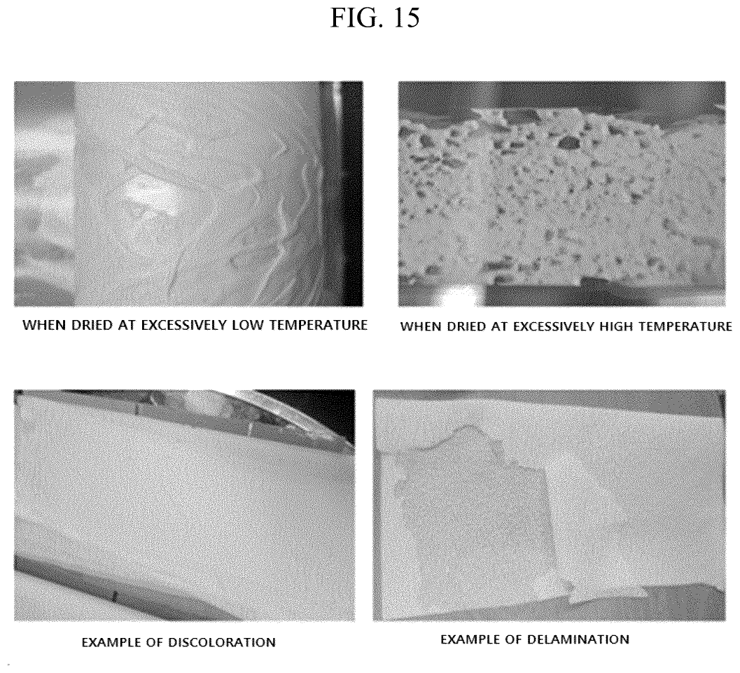

FIG. 1 illustrates an exemplary flowchart of a cosmetic coating method according to one embodiment of the present disclosure. FIG. 2 illustrates an overall schematic view of a cosmetic coating device according to one embodiment of the present disclosure. FIG. 3 illustrates an exemplary block diagram of the cosmetic coating device according to one embodiment of the present disclosure. FIG. 4 is an exemplary view of the cosmetic coating device from one side according to one embodiment of the present disclosure. FIG. 5 is an exemplary view of a support body supply part from one side according to one embodiment of the present disclosure. FIG. 6 is an exemplary view of a composition supply part from a side according to one embodiment of the present disclosure. FIGS. 7A to 7C are exemplary views of the composition supply part in various directions according to one embodiment of the present disclosure. FIG. 8 is an exemplary view illustrating an exterior of a solidifying treatment part according to one embodiment of the present disclosure. FIG. 9 is an exemplary view illustrating an inner side of the solidifying treatment part according to one embodiment of the present disclosure. FIG. 10 is an exemplary view of the solidifying treatment part from a rear according to one embodiment of the present disclosure. FIG. 11 is an exemplary view of a roll forming part from one side according to one embodiment of the present disclosure. FIG. 12 is an exemplary view of an embossing part from one side according to one embodiment of the present disclosure. FIGS. 13 and 14 are exemplary views for describing a bubble removing part according to one embodiment of the present disclosure. FIG. 15 shows results according to temperature conditions.

According to one embodiment, the present disclosure relates to a cosmetic coating device that is for manufacturing a cleansing sheet, and the coating device can mass-produce efficient cleansing sheets with consistent quality in rolls by applying a liquid composition at a predetermined thickness on a support body, suitably solidifying the liquid composition through light and temperature adjustment, and allowing the liquid composition to be well adhered to the support body.

In the present disclosure, “composition” may be a cosmetic raw material in the form of a liquid that includes an active ingredient providing main functionality of a cleansing sheet. A composition may include a moisturizing agent, a cleansing agent, an antibacterial agent, a fragrance, a preservative, or various natural extracts for soothing and improving the skin. The composition provides direct advantages to a user's skin and determines a main function of a cleansing sheet product.

In an embodiment, the support body may be a base medium for applying the composition and may be release paper, for example. Release paper may be a material in the form of a thin sheet having a non-adhesive surface that can be easily removed by a user to use the cleansing sheet after the composition is applied and cured. The release paper serves to support the composition in the process of applying and curing the composition and can be easily removed when the final product is applied on the user's skin.

The release paper is usually made of various synthetic plastic materials such as polyethylene, polypropylene, and polyester, and these materials have advantages that adhesion to the composition is low, chemical stability for a cosmetic composition is excellent, and mass production is possible at a low cost. The release paper also plays an important role in helping the user evenly distribute the composition while applying the cleansing sheet to the skin and easily discard the product after use.

In addition, in an embodiment, the support body may include a film, a biomass, and the like, but the support body is not limited thereto and may be made of various other materials. For example, the film may be configured to adjust a characteristic of adhesion to the composition, impart transparency, or provide a specific physical strength. The biomass may be used as a biodegradable material and may have an eco-friendly characteristic. In addition, materials of which the support body is made may be modified in various ways to include a natural fiber, a nonwoven fabric, a hydrophilic or hydrophobic coated substrate, a porous substrate, etc., and the materials may be appropriately selected according to characteristics, a purpose of use, a method of application, etc., of the composition. The constituents of the support body may be configured to maintain stability in the process of applying and curing the composition and finally provide a user-friendly product. FIG. 1 illustrates an exemplary flowchart of a cosmetic coating method according to one embodiment of the present disclosure. The order of steps illustrated in FIG. 1 may be changed when necessary, and at least one step may be omitted or added. The steps of FIG. 1 are only one embodiment of the present disclosure, and the scope of rights of the present disclosure is not limited thereto.

A cosmetic coating method according to the present disclosure may include applying a composition in a flowing state on one surface of a support body (S100).

In an embodiment, in the applying of the composition, the composition may be applied at a uniform thickness on the one surface of the support body using a gap between two or more rollers, and the applied composition may be irradiated with light of various wavelength bands for initial solidification of the applied composition using an initial curing module.

Specifically, in the applying of the composition, two or more rollers may be set in accordance with characteristics and a required coating thickness of the composition, and the composition may be uniformly applied on the one surface of the support body. In this case, the gap between the rollers may be adjusted according to viscosity and the required coating thickness of the composition, and in this way, the composition may be uniformly applied at a predetermined thickness on the support body. The application method utilizing the rollers has advantages that precise coating is possible, and air bubbles or nonuniform application, which may occur in the application process, can be minimized.

In addition, for initial solidification of the applied composition, the applied composition may be irradiated with light of various wavelength bands using the initial curing module. A light irradiation process may cause a chemical reaction to occur as a photoinitiator or a hardener in the composition absorbs light.

More specifically, an initial curing module 240 may include a plurality of light sources capable of emitting light of various wavelengths and may serve to optimize a process of curing the composition and stably solidify the composition within a short time. Light irradiation through the initial curing module 240 enables quick initial setting of the composition and provides a foundation for additional solidifying treatment in a subsequent process.

In an embodiment, the initial curing module 240 can be adjusted according to the type of the composition or a required degree of solidification of the composition and provides settings that can be adjusted by the user to provide versatility suitable for manufacturing various types of cleansing sheets. In addition, the initial curing module may have a structure capable of adjusting a separation distance between the light sources and a light irradiation angle in order to ensure uniform solidification of the composition.

The initial curing module 240 is provided to include a plurality of light sources radiating light of different wavelengths, and adjacent light sources are arranged to have different wavelength bands and promote uniform curing of the composition. Such arrangement may be a method in which a short wavelength light source is used to perform curing rapidly and a long wavelength light source is used to harden a deeper layer of the composition. This is to secure uniformity of curing by, for example, adjusting the amount of energy that each layer receives and allowing the entire composition to be uniformly cured.

In this way, the light sources of various wavelengths allow a multi-layer structure of the composition to be uniformly cured and allow light of different wavelengths to more effectively react with a specific chemical group or material in the composition, thereby optimizing the overall curing process. By combining the plurality of light sources, the initial curing module 240 may harden the composition in a customized manner in accordance with chemical characteristics and curing requirements of a specific composition. In this way, it is possible to provide effects of increasing efficiency of the manufacturing process and improving performance and quality of the final product. Through such a process, the applied composition undergoes curing to some extent in the initial step and undergoes an additional curing treatment process afterwards.

According to a specific embodiment, the applying of the composition may be performed through a composition supply part 200. The composition application process utilizing the composition supply part 200 will be described below with reference to FIGS. 2 to 11.

The cosmetic coating method according to the present disclosure may include performing curing treatment on the support body having the composition applied thereon (S200).

In an embodiment, the performing of the curing treatment may include inputting the support body having the composition applied thereon to one side of a solidifying treatment part and discharging the support body to the other side. According to one embodiment, the solidifying treatment part may perform curing of the composition located on one surface of the support body through control utilizing at least one of heat supply, hot air supply, cold air supply, and light supply.

In addition, in an embodiment, the performing of the curing treatment may include acquiring composition characteristic information corresponding to physical properties of the composition, determining, based on the composition characteristic information, a speed at which the support body having the composition applied thereon passes through the solidifying treatment part, and performing the curing treatment on the composition based on the determined speed.

First, the composition characteristic information corresponding to the physical properties of the composition may be acquired. The composition characteristic information may include a chemical composition, viscosity, reactivity with photocuring or thermosetting, a required curing time and temperature, etc., of the composition, and such information may be utilized in determining optimal treatment conditions of the composition in a coating process.

Next, based on the acquired composition characteristic information, the speed at which the support body having the composition applied thereon passes through the solidifying treatment part may be determined. The speed may be adjusted so that the composition can be irradiated with light for a sufficient amount of time for the composition to be completely cured. In various embodiments, several factors such as a curing time, a required light quantity of the composition, and a temperature condition of the solidifying treatment part may be considered in the process of determining the speed.

Lastly, the curing treatment on the composition is performed based on the determined speed. Specifically, the support body may be irradiated with light from a light irradiation module provided in the solidifying treatment part while passing through the solidifying treatment part, and the composition may be finally cured. In addition, efficiency of the curing process can be further improved by a temperature adjustment part maintaining the temperature in the solidifying treatment part to be suitable for the physical properties of the composition. Through such a curing treatment process, the composition may form a uniform, stable coating layer on the support body.

According to various embodiments, the applying of the composition may include determining, based on the composition characteristic information, an application thickness of the composition applied on the one surface of the support body and applying the composition on the one surface of the support body based on the determined application thickness.

In a specific embodiment, the composition characteristic information may be analyzed to determine a required application thickness. After the application thickness is determined, precisely applying the composition on the one surface of the support body based on the corresponding thickness may be performed. Then, in the application process, the composition may be coated at a uniform thickness on the support body. For example, in the case of roller coating, a gap between the rollers and the speed of the rollers may be adjusted in accordance with the physical properties of the composition, and the composition may be applied on the support body at the determined application thickness. Through such a process, the composition may form a coating layer having required physical and chemical characteristics.

According to various embodiments, the performing of the curing treatment may include determining, based on the composition characteristic information and the determined application thickness, the speed at which the support body having the composition applied thereon passes through the solidifying treatment part and performing the curing treatment on the composition based on the determined speed.

Specifically, in the performing of the curing treatment, first, the speed at which the support body should pass through the solidifying treatment part may be determined in consideration of the composition characteristic information and the thickness of the applied composition. The composition characteristic information may include a photocuring reaction speed, viscosity, and optical characteristics of the composition, and such information may be important factors in determining the light quantity that the composition needs and the time required for curing. In addition, the application thickness is also an important factor, and a thicker coating layer may require a larger light quantity or a longer curing time.

In a specific example, if the composition is based on a photocurable resin that reacts rapidly to UV light, the photocuring reaction speed of the composition may be relatively high. In this case, the speed at which the support body passes through the solidifying treatment part may be determined to be high, and sufficient curing may be possible with only a small quantity of UV light. On the other hand, if the composition being used has a high viscosity and a low photocuring reaction speed, the speed at which the support body passes through the solidifying treatment part may be determined to be low, and the composition may have to receive a larger quantity of light. Such a composition needs to be irradiated with light for a longer period of time, and in this way, the composition may be completely cured.

In addition, the larger the thickness of the applied composition, the larger the light quantity needed to reach all parts of the composition. For example, if a composition coating having a thickness of 100 μm is two times as thick as a coating having a thickness of 50 μm, a longer time and a larger light quantity may be needed for the entire composition to be sufficiently cured by light passing through the coating layer. In this case, the speed at which the support body passes through the solidifying treatment part may be adjusted to be low for sufficient light to reach the entire coated composition.

That is, in the present disclosure, the speed at which the support body passes through the solidifying treatment part may be determined in consideration of the characteristics and the application thickness of the composition, and in this way, the composition may be completely cured and form a high-quality coating layer.

With such a configuration, the speed at which the support body having the composition applied thereon passes through the solidifying treatment part may be determined. The support body passes through the light irradiation module inside the solidifying treatment part, and the curing treatment process is performed based on light of a wavelength band provided by the light irradiation module.

According to a specific embodiment, the performing of the curing treatment may be performed through a solidifying treatment part 300. The curing treatment process utilizing the solidifying treatment part 300 will be described below with reference to FIGS. 2 to 11.

The cosmetic coating method according to the present disclosure may include, after the curing treatment, producing a sheet using the support body on which the composition is solidified (S300). After the curing treatment, the support body on which the composition is solidified may be wound in one direction to produce a roll-shaped cleansing sheet.

In an embodiment, the process in which the support body on which the composition is solidified is wound in one direction to produce a roll-shaped cleansing sheet after the curing treatment may significantly improve efficiency in storage, transportation, and use of the product. This step is performed after the composition is completely cured, and the cured support body may be uniformly rolled to produce the form of the final product.

In the process of winding in a roll shape, the support body moves through a sheet forming part that is specially designed. The sheet forming part allows the support body to be uniformly wound while maintaining predetermined tension and may prevent the support body from excessively stretching or being damaged. Producing a roll-shaped cleansing sheet provides convenience also in a preparation process for packaging and shipping of the product, and in particular, since the roll-shaped cleansing sheet can be cut to a suitable length for use as necessary by the user, user convenience can be increased. That is, the roll-shaped cleansing sheet significantly improves flexibility and productivity and contributes to meeting various market needs.

According to a specific embodiment, the producing of the roll-shaped cleansing sheet may be performed through the solidifying treatment part 300. The curing treatment process utilizing the solidifying treatment part 300 will be described below with reference to FIGS. 2 to 11.

As illustrated in FIGS. 2 and 3, a cosmetic coating device 1000 of the present disclosure may include a support body supply part 100, the composition supply part 200, the solidifying treatment part 300, a sheet forming part 400, and a controller 500. The above components are illustrative, and the scope of rights of the present disclosure is not limited to the above components. That is, additional components may be included or some of the above components may be omitted according to implementation aspects of embodiments of the present disclosure.

According to one embodiment of the present disclosure, the cosmetic coating device 1000 unwinds a support body having a roll shape provided from the support body supply part 100 and applies a composition in a flowing state on one surface of the support body in a process of moving the support body in one direction.

The composition application process is performed by the composition supply part 200, and the composition is precisely applied at a uniform thickness on the surface of the support body. The support body having the composition applied thereon moves to the solidifying treatment part 300 afterwards, and a process of curing the composition is performed.

The solidifying treatment part 300 provides solidifying treatment in multiple steps by adjusting curing conditions such as light, humidity level, or temperature for the composition to be well adhered to the support body and have necessary physical properties. For example, the solidifying treatment part 300 may allow the composition to be suitably solidified through precise adjustment of light and temperature and may ensure quality and performance of the final product.

The support body that has passed through the solidifying treatment part 300 finally moves to the sheet forming part 400, and the sheet forming part 400 may wind the solidified support body in one direction and form a roll-shaped cleansing sheet. Through such a process, the cosmetic coating device 1000 may mass produce efficient cleansing sheets with consistent quality. According to the present disclosure, quality variation that may occur in the process of manufacturing a cleansing sheet can be minimized, and the stability and reliability of the product can be improved.

According to an embodiment, the cosmetic coating device 1000 may include the support body supply part 100 on which a support body roll having a roll shape is mounted. In an embodiment, the support body roll is mounted on the support body supply part 100 of the cosmetic coating device 1000 and continuously supplies the support body during a coating process. In this process, the support body roll is stably supported through a support body fixing part 120, and the support body fixing part 120 is supported in a direction perpendicular to the ground by a support plate 110. The support body fixing part 120 has a columnar shape for accommodating the support body roll and may be designed to pass through the center of the roll for the roll to be easily fitted and rotate. Through such a structural feature, the support body roll may be stably unwound during the coating process and may maintain predetermined tension necessary to uniformly apply the composition on the one surface of the support body.

In more detail, referring to FIGS. 4 and 5, the support body supply part 100 may include the support plate 110, the support body fixing part 120, and a support part 130. The support plate 110 has a plate shape and serves to support the support body fixing part 120. For example, the support plate 110 may be provided in the direction perpendicular to the ground, that is, the height direction, and may be provided to be connected to the support body fixing part 120 in a direction perpendicular to the direction in which the support plate 110 is provided (for example, the direction horizontal to the ground).

The support body fixing part 120 may be provided to extend from the support plate 110 and may be provided in a columnar shape that passes through the support body roll for the support body roll to be fitted thereto. For example, as illustrated in FIG. 5, the support body fixing part 120 may be provided in a cylindrical shape, and the support body roll is fitted to the support body fixing part 120. The user may easily mount the support body roll on the support body fixing part, and in this way, the support body is able to stably move within the cosmetic coating device. When the support body roll is fitted to the support body fixing part, the support body is unwound from the support body supply part, and the coating process starts.

In one embodiment, the support part 130 serves to support the support body unwound from the support body roll. The support part 130 allows the support body to stably move during the coating process and is an essential part for uniform application and treatment of the support body. The support part 130 may distribute a weight of the support body when the support body is unwound from the support body supply part 100 and may adjust tension of the support body to help the support body move at a suitable speed and in a suitable direction. Through the configuration of the support part 130, the support body is stably moved to the composition supply part 200, the solidifying treatment part 300, and the sheet forming part 400, and the overall operational efficiency and product quality of the cosmetic coating device 1000 can be improved.

In an embodiment, the structure and arrangement of the support part 130 may vary according to specific design of the cosmetic coating device 1000 and may be optimized in accordance with the width and weight of the support body and the degree of tension thereof necessary during the coating process.

According to one embodiment, the controller 500 may coordinate and optimize the overall operation of the cosmetic coating device. The controller 500 may precisely control various process parameters including an unwinding speed of the support body, an applied amount of the composition, an application thickness of the composition, parameters of the solidifying treatment (e.g., light intensity and temperature), and a speed of the roll forming process. In this way, conditions required in each process step are adjusted in real time, and process consistency and product quality are ensured.

For example, the controller 500 may be programmed through a user interface and may automatically adjust each process step according to parameters set by the user. In addition, the controller 500 may continuously monitor a state of a process through a sensor part and a feedback system and may, when necessary, automatically perform adjustment to maintain optimal operation conditions.

In an additional embodiment, the cosmetic coating device 1000 may include an input part receiving various input information from the user. An interface that allows the user to set various parameters related to the operation of the cosmetic coating device 1000 is provided. For example, the parameters may include an unwinding speed of the support body, an application thickness of the composition, curing conditions in the solidifying treatment part (e.g., temperature, light intensity and wavelength), and a roll forming speed. In an embodiment, composition characteristic information corresponding to the composition may be acquired through the input part. The composition characteristic information may include a chemical composition, viscosity, reactivity with photocuring or thermosetting, a required curing time and temperature, etc., of the composition, and such information may be utilized in determining optimal treatment conditions of the composition in a coating process. The user may input such composition characteristic information to the cosmetic coating device through the input part, and the controller may, based on the information, automatically adjust process parameters such as an unwinding speed of the support body, an application thickness of the composition, temperature and light conditions of the solidifying treatment, and a roll forming speed.

According to an embodiment, the user may finely adjust the coating process in accordance with desired requirements through the input part. For example, the input part may be implemented in various forms such as a touchscreen, a button, a dial, a keyboard, or a voice recognition function and may be designed in accordance with user convenience and process characteristics.

In an embodiment, the input part may transmit data received from the user to the controller, and the controller 500 may, based on the received information, suitably control each component of the cosmetic coating device 1000 to satisfy required production conditions.

According to one embodiment, the support body unwound from the support body roll may sequentially pass through the composition supply part 200 and the solidifying treatment part 300 and then be wound by the sheet forming part 400. In an embodiment, a rotation module for winding and unwinding may be provided on at least one of the support body supply part 100 where the support body roll is provided and the sheet forming part 400 where the support body on which the composition is applied and solidified is wound in a roll shape to produce a roll-shaped cleansing sheet. In an embodiment, the rotation module may be provided to include a motor controlling winding and unwinding speeds of the support body. The rotation module may be controlled by control of the controller 500. For example, the controller 500 may adjust the speed of the motor based on the parameters set by the user and may precisely control the winding and unwinding speeds of the support body.

According to one embodiment, the controller 500 may, based on the composition characteristic information, control an operation of at least one of the support body supply part 100 and the sheet forming part 400 so that the support body roll or the cleansing sheet is unwound or wound. The composition characteristic information may include a chemical composition, viscosity, reactivity with photocuring or thermosetting, a required curing time and temperature, etc., of the composition. Specifically, the controller 500 analyzes the composition characteristic information and adjusts the operation speed and time of the support body supply part and the sheet forming part. For example, if the composition has high viscosity and requires a longer time for curing, the controller 500 decreases the unwinding speed of the support body to allow the composition to stay on the support body for an adequate amount of time and be uniformly applied thereon for the composition to be sufficiently cured. Conversely, if the composition has low viscosity and curess rapidly, the controller 500 increases the unwinding and winding speeds to increase production efficiency.

In addition, if the composition shows an optimal curing reaction at a specific temperature, the controller 500 may adjust a temperature adjustment part 340 of the solidifying treatment part 300 and may control the composition to be cured under ideal conditions. In addition, in an embodiment, if the composition has reactivity to light of a specific wavelength, the controller 500 may adjust the initial curing module 240 and light source selection and irradiation intensity of a light irradiation module 330 to promote uniform curing of the composition.

In an embodiment, the support body unwound from the support body supply part 100 is supported by the support part 130, and afterwards, the support body moves to the composition supply part 200 and undergoes a process in which a composition in a flowing state is applied on one surface of the support body.

Referring to FIGS. 6 and 7, the composition supply part 200 may include a roller part 210 including two rollers provided to form a predetermined separation distance or more, a position adjustment part 212-2 adjusting a position of one of the two rollers to change a gap between the two rollers, a base part 220 guiding input of the composition toward the roller part 210, and the initial curing module 240 performing initial solidifying treatment on the applied composition when the composition is applied on one surface of the support body. The initial curing module 240 may be provided to be fixed to a holder 250. The holder 250 is a component for mounting the initial curing module 240 on one position and may be provided to correspond to one surface of a housing 300a. Here, the one surface of the housing 300a on which the holder 250 is provided may be one surface in which an inlet 300a-1 through which the support body that has passed through the composition supply part 200 is input is formed. The configuration in which the holder 250 is provided to correspond to the one surface of the housing 300a in which the inlet 300a-1 is formed helps the support body efficiently move from the composition supply part 200 to the initial curing module 240. Such arrangement may optimize a movement path of the support body and ensure consistency and efficiency of a process of irradiating the composition with light. In addition, such an arrangement structure facilitates maintenance, repair, and replacement tasks of the initial curing module 240 and allows replacement or adjustment to another light source to be easily performed when necessary. With such a configuration, the initial curing module 240 may effectively perform initial solidifying treatment on the composition applied on the support body after passing through the composition supply part 200. Since the initial curing module 240 is fixed to the holder and accurately positioned on a path of the support body having the composition applied thereon, the applied composition is able to receive radiated light. In the initial solidifying process through the initial curing module 240, the composition is stably adhered to the support body, and the composition is maintained in a suitable state in preparation for an additional curing process in the solidifying treatment part 300 afterwards.

According to one embodiment, the composition supply part 200 has the roller part 210 for uniformly applying the composition in a flowing state on the one surface of the support body and performs a function of applying the composition at a predetermined thickness on the one surface of the support body through the configuration of the roller part 210.

The roller part 210 included in the composition supply part 200 is configured to include two rollers, that is, a first roller 211 and a second roller 212, and since the two rollers remain spaced a gap 210a from each other, it is possible to uniformly apply the composition at a predetermined thickness on the one surface of the support body while passing through the support body. At this time, the separation distance between the rollers may act as an important factor that determines the thickness at which the composition is applied, and by adjusting this distance, the thickness of the applied composition can be precisely controlled.

The first roller 211 and the second roller 212 play an essential role in applying the composition on the surface of the support body. The first roller 211 may provide an initial contact point for delivering the composition to the support body, and the second roller 212 may perform a function of more uniformly spreading the composition while passing through the support body. The two rollers enable uniform distribution of the composition and application of the composition at a suitable thickness, and the gap between the rollers may be adjusted through the position adjustment part 212-2.

In an embodiment, referring to FIG. 7A, the two rollers may be provided to be fixed through a support 212-1. Specifically, the first roller 211 may be provided to be directly connected to the support 212-1, and the second roller 212 is provided on the support 212-1 through the position adjustment part 212-2 connected to the support 212-1. That is, the second roller 212 is provided to be connected to the support 212-1 via the position adjustment part 212-2. In this case, the position adjustment part 212-2 may be operated to change the position of the second roller 212 and adjust the gap from the first roller 211.

In an embodiment, the position adjustment part 212-2 may be configured in various forms such as a screw mechanism, a hydraulic or pneumatic system, or a fully automated motor driving system. In this way, the user or the controller 500 may precisely adjust the position of the second roller 212, and accordingly, the gap from the first roller 211 may be finely changed. For example, when the screw mechanism is utilized, the position adjustment part 212-2 may let the second roller 212 approach or move away from the first roller 211 through a rotating screw to adjust the gap between the rollers. When the hydraulic or pneumatic system is used, the second roller 212 is moved by adjusting pressure, and in the case of the fully automated motor driving system, precise position adjustment may be possible through rotation of an electric motor.

The position adjustment part 212-2 may provide flexibility that allows the application thickness of the composition to be adjusted in accordance with user requirements. In this way, when various types of support bodies or compositions are used in a production process, optimal application conditions may be promptly set in accordance with compositions having different physical properties. The precise adjustment function of the position adjustment part 212-2 plays a key role in maintaining consistency and quality of the product and may contribute to improving production efficiency. In an additional embodiment, the composition supply part 200 may also include a replaceable roller or application mechanism to apply various types of compositions.

In one embodiment, the controller 500 may control an operation of the position adjustment part based on the composition characteristic information and may adjust the gap between the two rollers. Specifically, the controller 500 may analyze the composition characteristic information and may adjust the gap between the two rollers in accordance with a required application thickness of the composition. For example, if the composition has high viscosity, since thicker application may be necessary, the controller 500 may operate the position adjustment part 212-2 and widen the gap between the two rollers so that the composition can be applied at a suitable thickness on the support body. Conversely, when the composition being used has low viscosity, since thinner application is possible, the controller 500 may reduce the gap between the rollers so that the composition can be applied thinly on the support body.

In addition, the controller 500 may control an operation of the position adjustment part 212-2 in consideration of information related to curing characteristics of the composition. For example, when a specific composition needs to be cured rapidly, the gap between the rollers may be narrowed to thinly apply the composition and accelerate the curing process. That is, the controller 500 may adjust the application thickness of the composition in accordance with a required curing speed of the composition, thereby controlling the composition to be cured under optimal conditions.

As described above, the controller 500 may allow uniform application and effective curing of the composition to be performed through precise gap adjustment between the rollers in consideration of chemical and physical characteristics of the composition.

That is, by adjusting the gap between the rollers through the position adjustment part 212-2, the user may easily change the thickness of the applied composition as necessary, and in this way, various requirements in the product production process may be easily satisfied.

In an embodiment, the base part 220 serves to guide the composition to effectively move to the roller part 210. To this end, the base part 220 is provided with a shape having an inclined surface. For example, the base part may provide an inclined surface to guide and optimize a flow of the composition when the composition moves to the roller part, and the inclined surface helps the composition naturally flow toward the rollers. Such a structure reduces waste of the composition and allows the composition to be uniformly applied on a predetermined portion of the support body, thereby contributing to improving the quality of the final product.

The base part 220 guides the composition in a required direction before reaching the roller part 210 and ensures stable supply and efficient application of the composition. The base part 220 may optimize a movement path of the composition to support a smooth flow to the roller part 210 and may minimize waste of the composition that may occur during the application process.

According to one embodiment, the composition supply part 200 may include a guide part 230 guiding the support body delivered from the support body supply part 100. Referring to FIG. 7B, the guide part 230 may be provided between the roller part 210 and the support body supply part 100 and may support and guide the support body delivered from the support body supply part 100 so that the support body is stably input to the composition supply part 200. The support body delivered from the support body supply part 100 passes the guide part 230, is guided to a lower end portion of the first roller 211, and then is discharged through a predetermined gap between the first roller 211 and the second roller 212. In this process, the support body is prepared for application of the composition, and the gap between the two rollers determines the exact thickness at which the composition will be applied on the surface of the support body.

In addition, the guide part 230 enables uniform application of the composition by allowing the support body to be aligned in a suitable direction and at a suitable position before entering the composition supply part 200. The design of the guide part 230 ensures that the support body is not distorted or wrinkled and minimizes errors that may occur in the composition application process to improve efficiency of the composition application process. That is, the guide part may optimize the movement path of the support body and may increase precision and consistency of the composition application process to ensure the quality of the final product.

According to an embodiment, the initial curing module 240 for initial solidifying treatment may be provided on the composition supply part 200. Specifically, right after the support body having the composition applied thereon passes between the two rollers, the initial curing module 240 serves to irradiate the composition with light and effectively solidify the surface and the inside of the composition.

In an embodiment, the initial curing module 240 is provided above the support body to perform initial curing treatment on the composition applied on the support body and is provided to include a plurality of light sources supplying light of different wavelength bands, and the light sources adjacent to each other are disposed to supply light of different wavelength bands.

More specifically, the initial curing module 240 may include a plurality of light sources capable of emitting light of various wavelengths and may serve to optimize a process of curing the composition and stably solidify the composition within a short time. Light irradiation through the initial curing module 240 enables quick initial setting of the composition and provides a foundation for additional solidifying treatment in a subsequent process.

In an embodiment, the initial curing module 240 can be adjusted according to the type of the composition or a required degree of solidification of the composition and provides settings that can be adjusted by the user to provide versatility suitable for manufacturing various types of cleansing sheets. In addition, the initial curing module may have a structure capable of adjusting a separation distance between the light sources and a light irradiation angle in order to ensure uniform solidification of the composition.

As illustrated in FIG. 7C, the initial curing module may be configured to include a plurality of light sources (for example, a first light source 241, a second light source 242, a third light source 243, and a fourth light source 244). The initial curing module 240 may include various light sources capable of emitting light of different wavelengths to optimize the process of curing the composition.

The initial curing module 240 is provided to include a plurality of light sources radiating light of different wavelengths, and adjacent light sources are arranged to have different wavelength bands and promote uniform curing of the composition. Such arrangement may be a method in which a short wavelength light source is used to perform curing rapidly and a long wavelength light source is used to cure a deeper layer of the composition. This is to secure uniformity of curing by, for example, adjusting the amount of energy that each layer receives and allowing the entire composition to be uniformly cured.

In a specific example, the first light source 241 may be configured as a UV light source and may serve to rapidly cure a surface layer of the composition. In this case, the first light source 241 is used for quick setting of the photocurable composition and accelerates an initial solidifying process of the composition. The second light source 242 is a light source within a visible light range and is suitable for curing an intermediate layer of the composition, and the third light source 243 may be configured as an infrared (IR) light source and may be utilized in uniformly curing a deeper layer of the composition. Additionally, the fourth light source 244 is a light source having a UV wavelength and may serve to promote uniform curing throughout the surface layer and inner layers of the composition together with the first light source 241. By arranging the light sources of different wavelengths in a zigzag pattern or another specific pattern, a light irradiation range is optimized, and light is uniformly distributed throughout the composition. Such a configuration may be very useful especially in the case of the photocurable composition when considering a depth at which light should reach and the amount of energy necessary for curing.

In this way, the light sources of various wavelengths allow a multi-layer structure of the composition to be uniformly cured and allow light of different wavelengths to more effectively react with a specific chemical group or material in the composition, thereby optimizing the overall curing process. By combining the plurality of light sources, the initial curing module 240 may cure the composition in a customized manner in accordance with chemical characteristics and curing requirements of a specific composition. In this way, it is possible to provide effects of increasing efficiency of the manufacturing process and improving performance and quality of the final product.

After the composition is applied on one surface of the support body, the support body having the composition applied thereon is delivered to the solidifying treatment part 300. The solidifying treatment part 300 serves to solidify the composition by performing a curing treatment operation on the support body on which the composition is applied by the composition supply part 200.

In one embodiment, the solidifying treatment part 300 may include the housing 300a forming an inner space 300b, the inlet 300a-1 formed in one surface of the housing 300a, an outlet 300a-2 formed in each of other surfaces corresponding to the one surface, a support surface 320 provided in the inner space 300b and supporting the support body input through the inlet from below, a light irradiation module 330 provided above the support surface 320, and the temperature adjustment part 340 changing a temperature of the inner space 300b.

In more detail, referring to FIGS. 8 to 10, the housing 300a of the solidifying treatment part 300 safely surrounds the inner space 300b and provides an environment for the solidifying treatment process of the composition. The inlet 300a-1 is formed in the one surface of the housing 300a to guide the support body, which has passed through the initial curing module 240, to the inside. One or more outlets 300a-2 are formed in one opposite surface or another side surface, and in this way, the cured support body is able to exit the solidifying treatment part 300 and move to a subsequent process.

In an embodiment, an input guide 310 may be provided at a front end of the inlet 300a-1. The input guide 310 serves to guide the support body having the initially cured composition applied thereon to the inside of the housing 300a. The input guide 310 may prevent the support body from being twisted or folded in a process of entering the solidifying treatment part 300 and may contribute to maintaining a state in which the composition is uniformly applied on the surface of the support body.

According to one embodiment, as illustrated in FIG. 4, the housing 300a may be provided to be supported at a predetermined height from the ground through a leg part 360, and accordingly, a space may be formed between the housing 300a and the ground. According to one embodiment, the support body supply part 100 and the sheet forming part 400 may be disposed in the space between the housing 300a and the ground.

Since the support body supply part 100 and the sheet forming part 400 are disposed in the space formed between the housing 300a and the ground, the overall design of the cosmetic coating device becomes compact, and efficient utilization of a working space becomes possible. Such a configuration improves accessibility of the device and further facilitates unwinding and winding processes of the support body. In addition, by utilizing the space between the housing and the ground, additional space can be secured for installing a necessary mechanical part or electric wiring, which facilitates maintenance and repair and service tasks of the device.

The structure in which the housing 300a is supported at a predetermined height from the ground through the leg part 360 also serves to improve stability of the device and may minimize an influence of vibrations, which may occur during an operation of the cosmetic coating device 1000, on the surrounding environment. This acts as an important factor in performing a precise coating process and can contribute to ensuring the quality of the final product.

As a result, the design in which the support body supply part 100 and the sheet forming part 400 are disposed in the space formed between the housing 300a and the ground can improve efficiency, accessibility, and stability of the device and can facilitate the operation, maintenance, and repair of the cosmetic coating device 1000.

According to an embodiment, the support surface 320 supporting the support body is provided in the inner space 300b of the housing 300a, and the support body passes above the support surface 320. The support surface 320 may stably support the support body when the support body passes below the light irradiation module 330 and may allow the support body to remain at a predetermined position in light irradiation and temperature adjustment processes.

According to one embodiment, the solidifying treatment part 300 may include an infrared light supply part provided on one surface of the support surface 320 to supply infrared light. Generally, in a drying facility, a target object (or composition) can be dried in various ways using a heater, steam, a hot air dryer, etc.

However, in the case of drying using a heater, steam, or hot air, low thermal efficiency and a low drying rate are pointed out as disadvantages, and various problems such as decreased productivity, external heat loss, high temperature inside the workplace, and taking up much space are caused.

In particular, in the case of the composition that is to be solidified after being applied on one surface of the support body according to the present disclosure, since the composition is a cosmetic raw material, uniform drying is not performed when drying of the inside is not suitably performed, and there is concern that a problem may occur in stability or ease of use of the finally produced cosmetic product.

In order to address such a problem, in the present disclosure, the infrared light supply part is provided in the solidifying treatment part 300 to adopt a drying method using infrared light. Drying using infrared light provides higher thermal efficiency and a higher drying rate compared to drying using a heater, steam, or hot air. In addition, the deep penetrating power of infrared light enables rapid heat transfer into the composition, enabling drying of the inside, which enables uniform solidifying and drying to be realized. Infrared light has a stronger thermal effect than visible light or UV light, and the principle of the thermal effect is that when infrared light is radiated and resonates with the chemical bond vibration energy of the composition, the infrared light is absorbed and converted into thermal energy inside the material into which it is absorbed. In this way, as the radiated infrared light is absorbed into the composition, infrared heating may be caused, which simultaneously generates heat on the surface and the inside of the material. That is, drying using infrared light can transfer energy at the speed of light even without a medium and can provide rapid, uniform, and highly efficient heating.

In addition, drying using infrared light can minimize heat loss and reduce a temperature increase inside the workplace, thereby improving productivity and a work environment. Further, an infrared drying device occupies relatively less space as compared to a general drying method (for example, a drying method using a heater, steam, or hot air), and its adaptable installation allows it to be easily integrated into various production lines.

According to a specific embodiment, the infrared light supply part of the present disclosure may supply near infrared (near-IR) light. In an embodiment, near-IR light may be infrared light having a wavelength band of 0.75 to 3 μm. More specifically, the near-IR light supplied by the infrared light supply part of the present disclosure may be infrared light having a wavelength band of 0.8 to 1.5 μm.

Since near-IR light has a shorter wavelength and a higher frequency than mid infrared light (for example, infrared light having a wavelength of 3 to 25 μm) and far infrared light (for example, infrared light having a wavelength of more than 25 μm), the near-IR light has high thermal penetrating power and can provide improved drying performance. When near-IR light is used, the near-IR light may deeply penetrate into a core of the composition and vaporize an evaporative substance inside a surface boundary, thereby allowing the composition to be dried from the inside to the outside. In this way, high drying efficiency and short work time can be guaranteed.

In summary, drying treatment specialized for cosmetic products can be supported by supplying infrared light in the inner space 300b, in particular, from below the support body having the composition applied thereon. In other words, by using infrared light, the composition can be more rapidly and uniformly solidified through high thermal penetrating power into the composition. In this way, production efficiency can be maximized while improving the stability and quality of the finally produced product.

In an embodiment, in a process in which the support body passes through the inside of the solidifying treatment part 300, the support surface 320 is provided below the support body, and the support surface 320 may be heated by a heating part 321. The solidifying treatment part 300 may further include the heating part 321 provided on an inner side of the support surface to supply heat. The heating part 321 may adjust a temperature of the inner space 300b and optimize the curing process of the composition. The heating part 321 uniformly supplies required heat while the support body passes the support surface 320, thereby allowing the composition to be suitably solidified at a required temperature. The heating part 321 may be operated using electrical resistance heating, infrared heating, or other heat sources and may maintain a specific temperature range in the solidifying treatment part 300 or may gradually adjust the temperature as necessary.

The heating function of the heating part 321 may be combined with light irradiation by the light irradiation module 330 and may optimize the curing process of the composition in various ways. Through such thermal and optical treatment, the composition on the support body may be uniformly solidified, and consistency and quality of the final product can be ensured. The heating part 321 may also operate in harmony with other components of the solidifying treatment part 300 and may promote rapid curing of the composition and improve production efficiency.

Meanwhile, the heating part 321 may be separately controlled for each area of the inner space 300b. A temperature of the heating part 321 located in a second area 300b-2 may be controlled to be the highest, and a temperature of the heating part located in a first area 300b-1 may be controlled to be lower than the temperature of the heating part in the second area. Lastly, the temperature of the heating part located in a third area 300b-3 may be controlled to be lower than the temperature of the heating part in the first area. The temperature of the heating part is controlled to be the highest in the second area because the second area is a portion where curing of the composition reaches a peak, and the temperature of the heating part is controlled to be the lowest in the third area among the three areas because the third area is a section where the cured composition is slowly cooled.

In an embodiment, the light irradiation module 330 may be provided above the support surface 320. In this case, the light irradiation module is provided to include a plurality of light sources radiating light of different wavelengths, and adjacent light sources are arranged to have different wavelength bands and promote uniform curing of the composition. Such arrangement may be a method in which a short wavelength light source is used to perform curing rapidly and a long wavelength light source is used to cure a deeper layer of the composition. This is to secure uniformity of curing by, for example, adjusting the amount of energy that each layer receives and allowing the entire composition to be uniformly cured.

In a specific embodiment, the light irradiation module 330 may be provided to include a plurality of light sources each corresponding to one of the plurality of areas inside the housing, and the light sources may be designed in a form in which near infrared (NIR) lamps and quartz infrared (QIR) lamps are alternately disposed. In this way, the arrangement of lamps (or light sources) with different wavelength bands allows energy to be uniformly transferred not only to the surface but also to the inside of the composition in a curing process of the composition and promotes effective curing of the entire composition.

In a specific embodiment, the light irradiation module 330 may be provided to include a plurality of light sources each corresponding to one of the plurality of areas inside the housing, and the light sources may be designed in a form in which near infrared (NIR) lamps and quartz infrared (QIR) lamps are alternately disposed. In this way, the arrangement of lamps (or light sources) with different wavelength bands allows energy to be uniformly transferred not only to the surface but also to the inside of the composition in a curing process of the composition and promotes effective curing of the entire composition.

An NIR lamp may have a wavelength of 2,200 to around 2,700 nm and may be effective in curing an inner part inside a surface of the composition. In addition, wavelengths in the above range are robust to a moisture absorption characteristic and thus can increase curing efficiency of the composition. A QIR lamp may have a wavelength of 700 to around 2,500 nm and may radiate light having a wavelength in a range of 900 to 1,500 nm, preferably. The QIR lamp serves to uniformly transfer heat to the composition.

According to an embodiment, the composition of the present disclosure relates to a cosmetic raw material and may include an aqueous base raw material. For example, in the present disclosure, the light irradiation module may be utilized to cause a chemical reaction in the aqueous base raw material and efficiently remove OH. The aqueous base may be a cosmetic composition that contains water as its main component, and OH may mean a hydroxyl group (—OH). The removal of OH from the aqueous base plays an important role in improving the stability and curing speed of the composition.

The removal of the hydroxyl group from the aqueous base cosmetic composition is promoted by light energy provided through the light irradiation module. Through a chemical curing method instead of a physical drying method, the physical and chemical properties of the composition can be optimized. Light energy reacts with the hydroxyl group and removes it, and as a result, curing of the composition can be performed more uniformly and rapidly. That is, through the chemical curing method using light energy instead of the physical drying method using hot air and heat supply, the physical and chemical properties of the composition can be optimized. The NIR and QIR lamps of the light irradiation module provide light of a suitable wavelength to each layer of the aqueous base composition and allow curing to be evenly performed from the surface to the inside.

For example, when the composition is cured through general hot-air drying, while the surface layer can rapidly cure as moisture rapidly evaporates from the surface, a problem in which the inner layer is not sufficiently cured may occur due to heat being unable to be effectively transferred to the inside of the thick composition. Effectively arranging and using the NIR lamp and the QIR lamp to address the above problem allows heat to be uniformly transferred to a deep layer of the composition and promotes a required chemical reaction and thus is very important in promoting uniform curing throughout the composition.

Due to its long wavelength characteristic, the NIR lamp may penetrate into deeper layers of the composition and induce a curing reaction inside, and the QIR lamp has a short wavelength characteristic and is effective in rapidly curing the surface of the composition. By alternately arranging the light sources of different wavelengths, a chemical reaction is caused to simultaneously occur inside and outside the composition, thereby making the overall curing process uniform.

Such arrangement of light sources may be adjusted in accordance with characteristics of each composition, for example, a chemical composition, a curing speed, a required heat quantity, etc., and through suitable spacing between the light sources and arrangement thereof, efficient curing of the entire composition is ensured while maintaining an optimal light irradiation range and intensity. This can greatly contribute to maximizing productivity while maintaining uniform product quality.

In summary, the solidifying treatment part 300 of the present disclosure may perform efficient curing of the composition by utilizing hot air, the light irradiation module, and heat supply. Specifically, curing of the surface of the composition may be performed through hot air supplied through the temperature adjustment part, curing of a middle end area of the composition may be performed through the light irradiation module, and curing of a lower area of the composition may be performed through the heating part 321 provided below the support body.

In this case, since the light irradiation module is provided in the form in which the light sources of different wavelength bands are alternately arranged, an upper portion of the middle area and a lower portion of the middle area may each be more efficiently cured.

In a specific example, when the composition has a first layer, a second layer, a third layer, and a fourth layer formed in the height direction, the first layer may be cured by hot air supply, the second layer may be cured by short wavelength light (for example, QIR light), the third layer may be cured by long wavelength light (for example, NIR light), and the fourth layer may be cured by the heating part.

In this way, the solidifying treatment part 300 of the present disclosure may provide uniform curing to all the areas of the composition, thereby improving the quality of the final product and maximizing physical performance thereof. By applying a different curing technique to each layer, curing may be performed in accordance with specific conditions that each area of the composition needs, and the overall durability and functionality of the product can be improved. Such a configuration may ensure uniform curing of the entire composition by preventing a situation in which only a specific area (for example, the surface) is cured due to a thickness of the composition and may effectively cause a reaction to occur even in inner layers to provide uniform curing in all the areas of the composition.

In addition, in an embodiment, since each layer of the composition is treated with a specific curing technique, all the layers may ultimately be uniformly cured, and optimal product quality can be ensured. The multi-layer curing approach of the present disclosure may be especially important because it allows each layer of the composition to have different chemical and physical properties. For example, the top layer may provide protection against the environment, the middle layer may provide structural strength, and the bottom layer may serve to increase durability of the product.

Since the curing process can be precisely controlled in accordance with requirements for each layer through the solidifying treatment part 300 of the present disclosure, it may be possible to manufacture a high-performance cosmetic product. In addition, such detailed curing control may contribute to reducing waste of materials and improving production efficiency.

As a result, by applying a curing technique suitable for each layer of the composition, the present disclosure enables development of a highly customized product and can provide an effective solution for satisfying needs in the cosmetic industry. Such a technical approach can maximize performance and stability of the product and can provide flexibility that allows various market needs to be met.

By combining the light sources of various wavelengths, the light irradiation module 330 may optimize a curing process extensively for various depths and ingredients of the composition. In addition, in an embodiment, by adjusting the light intensity and irradiation time of each light source through the controller 500, a customized curing profile may be set according to chemical properties and curing requirements of a specific composition. In this way, the entire composition may be uniformly cured, and the quality and performance of the final product may be improved.

In addition, in an embodiment, as illustrated in FIGS. 7A to 7C, the solidifying treatment part 300 may include the temperature adjustment part 340 supplying hot air or cold air. Specifically, the temperature adjustment part 340 may be provided to remove excessive heat that may be generated during a curing process or to provide a temperature condition necessary to adjust a curing speed of the composition. While the support body passes through the inner space of the housing 300a, the temperature adjustment part 340 may supply hot air or cold air to the inner space through a supply tube 341 to optimize a curing process of the composition. For example, hot air may be used in accelerating the curing of the composition, and cold air may be used in preventing overheating that may occur in the curing process and slowing down the curing speed when it is necessary according to characteristics of the composition.

In an embodiment, the temperature adjustment part 340 of the present disclosure may support cold air drying through cold air supply in addition to performing hot air supply. In one embodiment, there is concern that the composition to be solidified of the present disclosure may undergo a chemical change or deteriorate due to excessive heat when hot air drying is performed. For example, there is concern that structural stability may be damaged or active ingredients may be destroyed in a composition made of ingredients sensitive to high temperatures when hot air drying is performed. That is, cold air drying may have to be performed according to ingredients of a composition.

In a specific example, cold air drying of the present disclosure may be more usefully applied in a process of manufacturing a hydrogel mask. A hydrogel mask may include various active ingredients sensitive to heat, and it is important to avoid excessive heat to preserve efficacy of such ingredients. In the present disclosure, by supplying cold air through the temperature adjustment part in a drying process, the drying process can be promptly completed while maintaining the product quality and not causing the ingredients of the hydrogel mask to deteriorate due to heat.

That is, the temperature adjustment part 340 of the present disclosure serves to selectively adjust between hot air supply and cold air supply according to characteristics and requirements of a composition and optimize a solidifying treatment process of the composition. In particular, if the composition is sensitive to heat, cold air is supplied to prevent excessive heating of the composition during the curing process and to complete the solidifying treatment without deterioration of or damage to active ingredients. In this way, the stability and efficacy of the product can be maintained, and the final product with ensured quality can be produced. In addition, since the temperature adjustment part also has a function of supplying hot air to rapidly perform the curing process, flexibility and efficiency of the manufacturing process are significantly improved. Therefore, by providing drying and solidifying methods suitable for characteristics of various compositions, the present disclosure may contribute to expanding the product development scope and improving productivity.

Meanwhile, the temperature of hot air may also be separately controlled for each area of the inner space 300b. A temperature of hot air supplied to the second area 300b-2 may be controlled to be the highest, and a temperature of hot air supplied to the third area 300b-3 may be controlled to be lower than the temperature of the hot air supplied to the second area. Lastly, a temperature of hot air supplied to the first area 300b-1 may be controlled to be lower than the temperature of the hot air supplied to the third area. The temperature of the hot air supplied to the second area is controlled to be the highest because the second area is a portion where curing of the composition reaches a peak. Hot air at a higher temperature than the hot air supplied to the first area is supplied to the third area despite the third area being a cooling portion, and the third area may be controlled to be at a slightly lower temperature than the second area in consideration of the fact that physical properties of the composition may change when the temperature of the composition that has passed through the second area sharply decreases.

In one embodiment, the temperature adjustment part 340 may supply cold air after curing of the composition is completed. That is, the temperature adjustment part 340 may be provided to supply cold air to the cured composition at a discharge end of the solidifying treatment part 300. Here, the discharge end of the solidifying treatment part 300 may be a final outlet point through which the cured composition exits the solidifying treatment part.

Supplying cold air after curing is completed is important in promptly cooling the cured composition to reduce thermal stress and improving the dimensional stability of the product. The application of cold air prevents deformation or heat damage caused by excessive heat that may be generated in the process of curing the composition, thereby contributing to maintaining the quality of the final product. In addition, through the cooling process, it becomes easy to switch to subsequent treatment or a packaging process of the component, and the overall production cycle can be optimized.

According to an embodiment, the temperature adjustment part 340 is linked to a sensor part (for example, a temperature sensor) to enable the temperature of the support body to be monitored in real time, and in this way, the temperature of the space in which the support body and the composition are located may be precisely controlled.

Additionally, the solidifying treatment part 300 may further include a ventilation part for ventilating the inside air. The ventilation part may be provided through a discharge tube 342 discharging air and an air introduction fan. The ventilation part may remove excessive heat and moisture that may be generated during a process and may maintain optimal environmental conditions that are necessary while the composition is being cured. The discharge tube 342 of the ventilation part may effectively discharge the heat and moisture generated in the inner space to the outside, and the air introduction fan may improve indoor air quality and introduce required fresh air into the inner space. The ventilation part may serve to optimize the curing process of the composition and maintain uniform quality of the product.

With the configuration of the ventilation part, sharp changes in the temperature and the humidity level that may occur in the curing process may be adjusted, and a stable environment in which the composition can be uniformly cured can be provided. This may be more important particularly when the composition causes a sensitive chemical reaction or exhibits optimal performance only under specific temperature and humidity level conditions.

In addition, the ventilation part may also contribute to improving safety of the work environment. By effectively managing excessive heat and vapor of chemicals, health and safety of workers can be protected, and the air quality of the production space can be improved. In this way, work efficiency can be improved, and sustainability of the manufacturing process can be ensured in the long term.

In addition, in an embodiment, the inner space 300b of the solidifying treatment part 300 may be provided to be divided into a plurality of areas to perform control in different temperature ranges.

In one embodiment, an opening/closing member 350 may be formed on an outer side of the housing 300a. As illustrated in FIG. 8, the opening/closing member 350 may be formed to correspond to one surface of the housing and may be provided to be separately openable/closeable for each of the plurality of areas. The opening/closing member 350 may be located on the outer side of the housing 300a and may be opened or closed to make it easy to access the inside of the housing 300a as necessary. For example, when it is required to access the inside of the housing 300a for maintenance and repair, filling of the composition, or cleaning of the inside of the solidifying treatment part 300, the opening/closing member 350 may enable easy access to the inside of the housing 300a.

In one embodiment, the opening/closing member 350 may be implemented in various forms such as a slide type, a hinge coupling type, or a rollup type and may be implemented using a suitable method according to the form of the housing 300a and the use environment. In addition, the opening/closing member 350 provided to be separately openable/closeable for each area may allow a task to be performed only on a specific target area and may enable efficient work progress without affecting the overall process.

In one embodiment, the plurality of areas may include the first area 300b-1 controlled to 25° C. to 100° C., the second area 300b-2 controlled to 50° C. to 130° C., and the third area 300b-3 controlled to 50° C. to 110° C. Each of the areas may include a plurality of divided spaces according to the properties or size of an object to be heated.

In the first area 300b-1, initial solidification is provided to the composition for the composition to be stably attached to the support body, and the surface of the composition is smoothly solidified in the temperature range of 25° C. to 100° C. This is to increase the temperature to right below a boiling point of the aqueous composition, which is for preventing only the outer surface from burning, thoroughly supplying heat, and preventing the outside and inside from being separated. In a specific embodiment, it may be most preferable to maintain the temperature of the first area in a range of 40 to 60° C.

Then, in the second area 300b-2, the composition is deeply cured to a higher temperature ranging from 50 to 130° C. to increase durability and adhesion. Since, if the temperature is raised too high, the sheet will react, causing bubbles to form or heat to build up, this is prevented and curing is allowed to sufficiently occur inside in the second area 300b-2. In a specific embodiment, it may be most preferable to maintain the temperature of the second area in a range of 90 to 110° C.

Lastly, in the third area 300b-3, a final curing process of the composition is performed at a temperature controlled to be in a range of 50 to 110° C. to stabilize the quality of the final product. A reason for gradually lowering the temperature is to further improve structural stability, and due to characteristics of the aqueous composition, the final curing process is performed to reach almost 100° C. While performing curing at a temperature close to 80° C. is preferable for a composition containing an oil, performing curing at a temperature close to 100° C. is advantageous in other cases. That is, it is preferable to control the temperature of the third area to 80 to 100° C.

In this way, the solidifying treatment part 300 of the present disclosure can enhance the quality of the final product and maximize physical performance thereof by providing uniform curing of the composition through temperature control suitable for each area.

As illustrated in FIG. 9, the first area 300b-1, the second area 300b-2, and the third area 300b-3 are arranged sequentially, and the support body sequentially passes through the areas and undergoes a process in which the composition is gradually cured. Such sequential arrangement allows the curing process of the composition to be efficiently managed and enables precise temperature control required for each step, thereby ensuring consistent quality and performance of the final product.

According to one embodiment, the plurality of areas may each have a predetermined length. For example, a length of each area may be 6 meters. In this case, the support body having the composition applied thereon may move 6 meters per minute and may stay 1 minute in each area. That is, curing is performed for a total of 3 minutes on the support body, and after 3 minutes, curing is completed, and the support body is discharged. Specific numerical values relating to the length of each area and the movement speed stated above are only examples, and the present disclosure is not limited thereto.

According to one embodiment, the solidifying treatment part 300 may include a sensor part sensing environment information of the inner space. The sensor part may serve to monitor environmental conditions inside the solidifying treatment part 300, such as a temperature, a humidity level, light intensity and wavelength, in real time. The environment information acquired through the sensor part greatly affects the solidifying process of the composition and thus is utilized by the controller 500 for the composition to be cured under optimal conditions. For example, when the sensor part detects that the temperature has deviated from a target range, the controller 500 may adjust the temperature adjustment part 340 to adjust the temperature of the inner space. In another example, when the light intensity is determined to be insufficient for curing of the composition, the controller 500 may increase output of the light irradiation module and supply a required light quantity. The sensor part allows a problem that may occur during a process to be detected at an early stage and handled, thereby contributing to increasing process efficiency and improving productivity.

According to one embodiment, the solidifying treatment part 300 may include a separation membrane dividing the inner space into the plurality of areas. The separation membrane may be provided to change a size of a movement passage between adjacent areas. In an embodiment, each separation membrane may form a movement passage through which the support body having the composition applied thereon is able to pass, and the support body having the composition applied thereon may be cured while sequentially moving along each area through the movement passage formed by each separation membrane.

In an embodiment, the controller 500 may control an operation of the separation membrane based on environment information of each of the plurality of areas. Specifically, the controller 500 may analyze environment information collected from the sensor part, for example, a temperature, a humidity level, a light intensity, and the like of each area, and may optimize conditions of each area. For example, when curing conditions of the composition are determined to not be ideal in a specific area, the controller 500 may adjust the position of the separation membrane to adjust the size of the movement passage between the corresponding area and an area adjacent thereto. In this way, the curing process of the composition may be optimized by adjusting a flow of heat or moisture from the adjacent area or adjusting a light irradiation range.

For example, when curing of the composition is slowly performed in a specific area, the controller 500 may adjust the separation membrane so that more heat is transferred to the corresponding area. Conversely, when curing is performed too rapidly and there is a risk that the composition may not be set properly, the controller 500 may adjust the separation membrane to lower the temperature of the corresponding area or decrease the light intensity.

Such operation control of the separation membrane may allow the curing process of the composition to be managed in more detail in the solidifying treatment part and may contribute to increasing process flexibility and product quality consistency.

In a specific example, when the curing temperature of the composition is below a certain range in the first area, the controller 500 may adjust the separation membrane and widen the movement passage between the first area and the second area adjacent thereto so that more heat is transferred to the first area. As a result, the temperature of the first area increases, and the curing speed increases. Conversely, when curing is performed too rapidly and the composition is excessively cured in the third area, the controller may adjust the separation membrane to narrow or block the movement passage of the third area so that transfer of heat and light is reduced. In this way, by lowering the temperature of the third area or decreasing the light intensity, the composition is allowed to harden at a suitable speed.

In this way, the operation control of the separation membrane optimizes curing conditions of the composition and adjusts the environment of each area in detail, thereby allowing the composition to be uniformly cured as a whole. This may contribute to increasing process flexibility and maintaining consistency in product quality.

In addition, by adjusting the flows of heat and moisture between different areas through the separation membrane, a curing problem that may occur in a specific area may be promptly addressed. For example, when a humidity level is high and the composition is not cured properly in a specific area, the separation membrane may be adjusted so that dry air flows to the corresponding area. Conversely, when a light irradiation intensity is too high and the composition is rapidly cured in a specific area, the transfer of light may be blocked or reduced through the separation membrane.

In this way, the solidifying treatment part 300 of the present disclosure can maximize process efficiency and product quality by precisely controlling the curing process of the composition through the separation membrane and the controller 500.

In addition, according to an embodiment, the cosmetic coating device 1000 may include the sheet forming part 400 forming a roll-shaped cleansing sheet by winding the support body, on which the composition is solidified after passing through the solidifying treatment part 300, in one direction.

The sheet forming part 400 may serve to effectively wind the support body, on which the composition is solidified after passing through the solidifying treatment part, in a roll and form a final cleansing sheet product. The sheet forming part 400 may include a precise rolling mechanism to roll the support body at a predetermined speed and with uniform tension. The sheet forming part 400 also has an adjustment function for adjusting the size and length of the roll to correspond to user requirements, and in this way, a cleansing sheet product can be produced in various sizes and lengths.

According to one embodiment, the sheet forming part 400 may include a roll fixing part 410 and a roll guide part 420. The roll fixing part 410 may serve to fix a roll. Here, the roll may be a cylindrical structure around which the final cleansing sheet product is wound. The roll may be provided to have a hollow center and be fitted to the roll fixing part 410. That is, the roll having a cylindrical shape may be fixed through the roll fixing part 410. The roll is inserted into the roll fixing part 410 through a central hole inside the roll and is stably located.

According to an embodiment, the roll fixing part 410 may be provided to be rotatable. Specifically, the roll fixing part 410 may be configured to include a roller bearing, a shaft, or other rotation support structures. Such components may support the roll to smoothly rotate and be stably fixed to a necessary position during a coating process. Meanwhile, a rotation module may be provided on one surface of the roll fixing part 410. The rotation module may apply a rotational force for rotating the roll fixing part 410. When the roll fixing part 410 rotates, the roll fitted to the roll fixing part rotates, and accordingly, the support body on which the composition is solidified is wound around the corresponding roll. A rotational force may be applied to the roll fixing part 410 through the rotation module, and in this way, as a rotational speed of the roll is adjusted, a speed at which the support body passes through the cosmetic coating device 1000 (or the solidifying treatment part) may be determined. For example, when the rotational speed is increased, the support body may be wound more rapidly around the roll, and this may cause the support body to more rapidly pass through the solidifying treatment part of the cosmetic coating device 1000 and may shorten the curing time of the composition. Conversely, when the rotational speed is decreased, the support roll may be wound more slowly around the roll, and this may cause the composition to stay longer on the roll before solidifying and may slow down the curing process. The rotation module may be operated by control of the controller. According to an embodiment, the controller 500 may control the operation of the rotation module based on composition characteristic information. For example, if a composition having high viscosity is used, the controller may decrease the rotational speed so that the composition is sufficiently delivered to the roll and is uniformly coated. Conversely, if a composition having low viscosity is used, the controller may increase the rotational speed to prevent excessive accumulation of the composition and accelerate the curing process.

That is, the controller 500 may precisely adjust the rotational speed and time of the rotation module based on the viscosity and density of the composition and information on other physical and chemical properties of the composition.

The roll guide part 420 serves to guide the support body on which the composition is solidified (that is, the support body that has passed through the solidifying treatment part) to be stably wound around the roll mounted on the roll fixing part 410. The roll guide part 420 may support the support body and guide the support body to move along a predetermined path without shaking. In an embodiment, the roll guide part 420 may be provided as a plurality of roll guide parts 420. For example, the roll guide part 420 may include a first roll guide part 421 and a second roll guide part 422. The first roll guide part 421 and the second roll guide part 422 may be provided so that the support body can be stably wound around the roll while maintaining the predetermined path during a process of conveying the support body. Shaking or detachment of the support body may be prevented by using the plurality of roll guide parts. According to an embodiment, the accurate arrangement and adjustment of the first roll guide part 421 and the second roll guide part 422 may optimize the conveying path and speed of the support body.

That is, the roll guide part 420 may ensure stable conveying and accurate position adjustment of the support body. The roll guide part 420 may support both ends of the support body and may guide the support body to be evenly wound around the roll fitted to the roll fixing part 410 at a predetermined speed. The guide part 230 guides linear movement of the support body and adjusts an angle and tension of the support body relative to the roll so that the support body maintains uniform thickness and quality even after passing through the solidifying treatment part 300.

In addition, the position of the roll guide part 420 may be adjusted to vary according to characteristics of the composition and requirements of the final product. For example, the position or angle of the roll guide part 420 may be adjusted when treating a thicker support body or a support body having different physical properties, and accordingly, the conveying path of the support body may be optimized. Such a configuration can improve efficiency of the production process while maintaining consistency in product quality.

In various embodiments, the sheet forming part 400 may include a guide roller or a tensioner for minimizing wrinkles or deformations that may be generated in a process of winding the support body in a roll shape. Accordingly, the quality of the final product may be ensured. In addition, the sheet forming part 400 may be provided to further include an additional module that can perform an additional function such as applying an additional protective layer on both surfaces of the support body, on which the composition is solidified, as necessary in a roll forming process or adding labeling for product identification.

The sheet forming part 400 also plays an important role in a preparation process for packaging and shipping of the product and improves convenience in use of the product by allowing the roll-shaped cleaning sheet to be cut to a suitable length for use by a user as necessary. The inclusion of the sheet forming part 400 can significantly improve flexibility and productivity of the cosmetic coating device 1000 and can contribute to meeting various market needs.

In an additional embodiment, the cosmetic coating device 1000 of the present disclosure may include an embossing part 600 forming grooves of a specific pattern on the support body on which the composition is applied and solidified. In one embodiment, the embossing part 600 may be provided between the solidifying treatment part 300 and the sheet forming part 400 and may form grooves of a specific pattern on the support body on which the composition is applied and solidified.

According to an embodiment, as illustrated in FIG. 12, the embossing part 600 may include an embossing guide 610, a main body part 620, a pattern roller housing 630a, a pattern roller 630, a bottom surface 640, and a roller position adjustment part 622.

The embossing guide 610 serves to guide the support body having one surface on which the composition is applied and solidified that is discharged through the outlet of the solidifying treatment part 300 toward the pattern roller 630. The embossing guide 610 ensures that the support body accurately passes through the pattern roller, thereby enabling precise pattern formation.

In addition, the main body part 620 provides a structural frame of the embossing part 600 and supports the pattern roller housing 630a. The pattern roller housing 630a is fixed to the main body part 620 and allows the pattern roller 630 to be stably located, thereby enabling a pattern to be applied to the support body.

The pattern roller 630 is a roller with a specific pattern engraved thereon, and when the support body on which the composition is applied and solidified passes through a space between the pattern roller 630 and the bottom surface 640, grooves of a desired pattern may be formed on the support body.

According to an embodiment, the pattern roller 630 and the bottom surface 640 may remain spaced at a predetermined distance, and the distance may determine a depth of the pattern applied to the support body.

In one embodiment, the roller position adjustment part 622 may change the size of the space between the pattern roller 630 and the bottom surface 640 by changing the position of the pattern roller housing 630a relative to the main body part 620.

In a more specific example, the roller position adjustment part 622 may adjust a vertical position of the pattern roller housing 630a relative to the main body part 620 to finely adjust the separation distance between the pattern roller 630 and the bottom surface 640. In this way, the depth of the pattern applied to the support body on which the composition is applied and solidified may be precisely set. For example, when it is desired to form a deeper pattern on the support body, the pattern roller housing 630a may be moved downward through the roller position adjustment part 622 to decrease the distance between the pattern roller 630 and the bottom surface 640. Conversely, when it is desired to form a shallower pattern, the pattern roller housing 630a may be moved upward to increase the distance between the pattern roller 630 and the bottom surface 640.

Such an adjustment function allows product diversity and customized design needs to be met and plays an important role in improving final product quality and user satisfaction. The adjustment ability of the roller position adjustment part 622 can significantly improve flexibility of the manufacturing process and allows customized patterns suitable for characteristics and requirements of various cosmetic products to be promptly and precisely applied.

In summary, the embossing part 600 may precisely form grooves of various depths and patterns on the support body on which the composition is applied and solidified in the cosmetic coating device. The pattern formed on the support body may be designed in various ways to improve product functionality, aesthetics, and user experience.

In an embodiment, the pattern may also serve to enhance safety of the product. For example, a slip prevention pattern prevents slippage during use of the product and reduces a risk of accidents that may occur during use. Such a functional pattern provides a safer and more comfortable user experience to the user. In addition, the pattern may serve to adjust a release speed of active ingredients.

In addition, the pattern may include an aesthetic element such as a visually pleasing design or a logo for brand identification and also contributes to improving sensory satisfaction in a process in which the user uses the product. The visual element of the pattern may also play an important role in product packaging or branding of the product itself and may help a consumer easily recognize and remember the brand. This is part of a marketing strategy and may contribute to attracting consumers' interest through visual appeal of the product and strengthening the brand image.

Additionally, a specific pattern formed through the embossing part 600 may also play an important role in improving convenience in use of the product. For example, a pattern including dotted lines or specially designed cutting lines that facilitate splitting makes it easy for the user to adjust the product to a desired size or shape. This is useful especially in a multipurpose cosmetic product or a product that requires amount adjustment.

In summary, the embossing part 600 performs an essential function in the cosmetic coating device 1000 and may precisely form various patterns improving functionality, aesthetics, and user experience on the support body. The pattern may contribute to improving usability and safety of the product and identifiability of the brand and may ultimately improve consumer satisfaction and strengthen market competitiveness.

In an additional embodiment, the cosmetic coating device 1000 may include a bubble removing part 700.

In one embodiment, the bubble removing part 700 may effectively remove bubbles in a sheet manufacturing process performed in a roll-to-roll manner and may improve product quality. Although the roll-to-roll manner is generally advantageous for mass production because continuous production is possible, there are problems that bubbles are likely to be included in a process of applying a liquid cosmetic formulation, and the bubbles expand or condense while undergoing a heating process, in particular. The bubbles may disrupt uniformity of a sheet surface and may affect functional performance as well as aesthetic quality of the final product. Accordingly, in the present embodiment, the bubble removing part 700 is applied to effectively remove unnecessary bubbles during a process.

For example, in a sheet production process, as an applied liquid formulation is heated, the air contained therein may remain in the form of bubbles without escaping in some cases. In particular, in a process of manufacturing a support body type cosmetic patch or sheet mask, since a formulation is required to be thinly spread and uniformly coated, residual bubbles may be a more serious problem. In addition, in a cosmetic formulation containing a specific ingredient, bubbles may easily form but not be easily removed due to a difference in surface tension, and the bubbles may remain trapped in a coating layer and act as a factor that decreases product consistency. In order to address this, in the present embodiment, a metal bar 720 may be used to physically guide and remove bubbles in the applied formulation, and an electrical method may be applied as necessary to promote bubble decomposition.

In a specific example, in a process of producing an aqueous cosmetic sheet, a method in which a liquid mixture of an aqueous polymer and a cosmetic material is applied on a continuous support body and then heated to be solidified is generally applied. However, bubbles formed in this process are a main cause of surface nonuniformity of the final product. The aqueous cosmetic sheet is a cosmetic material that has a characteristic of easily melting in a specific solvent and is designed to naturally dissolve after a certain period of time when adhered onto the skin. Due to such a characteristic, it is very important for the sheet to maintain a predetermined thickness and uniform surface quality, but when bubbles are included in the production process, the surface may roughen or transparency may decrease. In particular, the aqueous cosmetic sheet has higher surface tension than a polyvinyl alcohol (PVA)-based sheet and thus natural bubble removal is difficult, and there is concern that bubbles may further condense and expand during a heating process. While bubbles easily spread and rapidly disappear in the PVA-based sheet due to relatively low surface tension, bubbles are likely to remain inside the aqueous cosmetic sheet due to the aqueous cosmetic sheet being made of a highly viscous aqueous polymer.

Such a problem may lead to a decrease in tactile quality in addition to simply decreasing exterior quality. For example, a sheet solidified while containing bubbles may provide an uneven feeling of touch when the user attaches the sheet to the skin, and this may be a factor that weakens competitiveness of a high-end cosmetic product. Since soft texture and uniform surface quality are some important factors that consumers consider when selecting a premium product, addressing the bubble problem is essential in increasing product value. In addition, addressing the bubble problem may also affect brand reliability and may be an important factor in getting consumers to repeatedly purchase the product.

Accordingly, in the present disclosure, a bubble removing part utilizing an electrostatic field to effectively remove bubbles is applied to a production process, thereby enhancing the surface quality of the aqueous cosmetic sheet and improving efficiency of the manufacturing process.

Meanwhile, it is required to add a chemical additive or adopt a separate vacuum process to remove bubbles in a conventional manner, but this may lead to an increase in process costs and a decrease in production efficiency. In particular, the use of chemical additives is limited because it may be associated with safety and regulatory compliance issues for cosmetic ingredients.

In the present embodiment, by applying the bubble removing part 700 that can effectively remove bubbles in a physical manner, a cosmetic sheet with uniform quality can be manufactured without a separate chemical additive.

The present disclosure may include the bubble removing part 700 removing bubbles in order to address the above-described bubble formation problem.

According to an embodiment, as illustrated in FIG. 13, the bubble removing part 700 includes a bubble support bar 710 and the metal bar 720, and the metal bar 720 may be configured so that electricity can flow therein and may perform a function of electrostatically removing bubbles.

Referring to FIG. 13, bubbles may be formed in various sizes in a formulation during a coating process, and the formed bubbles may gather into larger bubbles over time or expand according to temperature changes and may form a nonuniform pattern on a sheet surface. In particular, in an aqueous polymer-based cosmetic formulation such as an aqueous cosmetic sheet, since surface tension is high, bubbles are likely to remain without disappearing naturally. In order to address such a problem, the bubble removing part 700 is disposed to remove bubbles before a liquid formulation containing bubbles enters a heating process (for example, enters the solidifying treatment part) during the roll-to-roll process.

Referring to FIG. 14, the metal bar 720 of the bubble removing part 700 may play a role in bubble removal. The metal bar 720 forms a predetermined electrostatic field due to power supplied thereto, and in this way, negatively charged bubbles in the formulation may be guided toward the metal bar 720. That is, bubbles may be removed by being dispersed or adsorbed onto the metal bar 720 due to an electrical attractive force. The metal bar 720 is disposed at a position adjacent to the applied liquid formulation (for example, at a distance of about 3 mm therefrom), and the distance may be adjusted according to a bubble formation situation. That is, since an electrostatic effect of the metal bar 720 causes bubbles that are negatively charged due to a cosmetic ingredient in the formulation to be drawn to the metal bar and removed, bubbles can be removed from the sheet.

Meanwhile, the bubble support bar 710 serves to stably maintain the metal bar 720 and contributes to forming a uniform electric field in a bubble removal process. In addition, the bubble support bar 710 has a structure that provides reinforcement to allow the metal bar 720 to remain at a predetermined position even during a continuous process in a roll-to-roll manner.

FIG. 15 shows results according to temperature conditions. When a curing task is performed under a temperature condition lower than a designated temperature condition in the solidifying treatment part 300, the composition is less cured or delamination occurs. On the other hand, when a curing task is performed under a temperature condition higher than a designated temperature condition, defects such as the composition not curing evenly, cracking, or hole formation occur. Meanwhile, delamination is more prominent when an NIR lamp is not used or temperature control of the heating part 321 is not performed under optimal conditions.

Embodiments of the present disclosure have been described above with reference to the accompanying drawings, but those of ordinary skill in the art to which the present disclosure pertains should understand that the present disclosure may be carried out in other specific forms without changing the technical spirit or essential features thereof. Therefore, the embodiments described above should be understood as illustrative, instead of limiting, in all aspects.

Specific executions described in the present disclosure are embodiments, and the scope of the present disclosure is not limited to specific methods. For simplicity of the specification, description of conventional electronic components, control systems, software, and other functional aspects of the systems may be omitted. As examples of connections of lines or connecting elements between the components illustrated in the drawings, functional connections and/or physical or circuit connections are exemplified, and in actual devices, those connections may be replaced with other connections, or may be represented by various additional functional connections, physical connections or circuit connections. Furthermore, unless specifically defined using the term “essential,” “significantly” or the like, the corresponding component may not be an essential component required in order to apply the present disclosure.

It should be understood that the specific order or hierarchy of steps in the processes disclosed herein is an example of exemplary approaches. Based upon design preferences, it is understood that the specific order or hierarchy of steps in the processes may be rearranged while remaining within the scope of the present disclosure. The accompanying method claims present elements of the various steps in a sample order, and are not meant to be limited to the specific order or hierarchy presented.

The description of the embodiments disclosed herein is provided to enable those of ordinary skill in the art to make or use the present disclosure. Various modifications to these embodiments will be readily apparent to those skilled in the art, and the generic principles defined herein may be applied to other embodiments without departing from the scope of the present disclosure. Thus, the present disclosure is not intended to be limited to the embodiments shown herein but is to be accorded the widest scope consistent with the principles and novel features disclosed herein.

(Description of reference numerals)

| | |
|---|---|
| 1000: cosmetic coating device | |
| 100: support body supply part | 110: support plate |
| 120: support body fixing part | 130: support part |
| 200: composition supply part | 210: roller part |
| 211: first roller | 212: second roller |
| 210a: gap | 212-1: support |
| 212-2: position adjustment part | 220: base part |
| 230: guide part | 240: initial curing module |
| 241: first light source | 242: second light source |
| 243: third light source | 244: fourth light source |
| 250: holder | |
| 300: solidifying treatment part | 300a: housing |
| 300a-1: inlet | 300a-2: outlet |
| 300b: inner space | 300b-1: first area |
| 300b-2: second area | 300b-3: third area |
| 310: input guide | 320: support surface |
| 321: heating part | 330: light irradiation module |
| 340: temperature adjustment part | 341: supply tube |
| 342: discharge tube | 350: opening/closing member |
| 360: leg part | 400: sheet forming part |
| 410: roll fixing part | 420: roll guide part |
| 421: first roll guide part | 422: second roll guide part |
| 500: controller | 600: embossing part |
| 610: embossing guide | 620: main body part |
| 622: roller position adjustment part | 630: pattern roller |
| 630a: pattern roller housing | 640: bottom surface |
| 700: bubble removing part | 710: bubble support bar |
| 720: metal bar | |

### MODES OF THE INVENTION

The relevant content has been described above in the best mode of the invention.

1. A cosmetic coating device comprising:
a composition supply part applying a composition in a flowing state on one surface of a support body;
a solidifying treatment part curing the support body having the composition applied thereon by the composition supply part; and
a sheet forming part manufacturing a sheet using the support body having the composition applied thereon.

2. The cosmetic coating device of claim 1, further comprising:
a support body supply part on which a support body roll having a roll shape is mounted; and
a controller controlling operations of the composition supply part, the solidifying treatment part, and the sheet forming part,
wherein the support body unwound from the support body roll sequentially passes through the composition supply part and the solidifying treatment part and is wound in the sheet forming part.

3. The cosmetic coating device of claim 2, wherein the composition supply part includes:
a roller part including two rollers provided to form a predetermined separation distance or more;
a position adjustment part adjusting a position of one of the two rollers to change a gap between the two rollers;
a base part guiding input of the composition toward the roller part; and
an initial curing module performing initial solidifying treatment on the applied composition when the composition is applied on the one surface of the support body.

4. The cosmetic coating device of claim 3, further comprising an input part acquiring composition characteristic information corresponding to the composition,
wherein the controller controls an operation of at least one of the support body supply part and the sheet forming part so that the support body roll or the sheet is unwound or wound based on the composition characteristic information.

5. The cosmetic coating device of claim 4, wherein the controller controls an operation of the position adjustment part based on the composition characteristic information to adjust the gap between the two rollers.

6. The cosmetic coating device of claim 3, wherein the initial curing module is provided above the support body to perform initial curing treatment on the composition applied on the support body and is provided to include a plurality of light sources supplying light of different wavelength bands, and the light sources adjacent to each other are disposed to supply light of different wavelength bands.

7. The cosmetic coating device of claim 2, wherein the solidifying treatment part includes:
a housing forming an inner space;
an inlet formed in one surface of the housing;
an outlet formed in each of other surfaces corresponding to the one surface;
a support surface provided in the inner space and supporting the support body input through the inlet from below;
a light irradiation module provided above the support surface; and
a temperature adjustment part changing a temperature of the inner space.

8. The cosmetic coating device of claim 7, wherein the inner space is provided to be divided into a plurality of areas to perform control in different temperature ranges and includes:
a first area in which a temperature is adjusted to be in a range of 25° C. to 100° C.;
a second area in which a temperature is adjusted to be in a range of 50° C. to 130° C.; and
a third area in which a temperature is adjusted to be in a range of 50° C. to 110° C.

9. The cosmetic coating device of claim 7, wherein the solidifying treatment part further includes:
a sensor part sensing environment information of the inner space; and
a heating part provided on an inner side of the support surface to supply heat.

10. The cosmetic coating device of claim 8, wherein:
the solidifying treatment part includes a separation membrane dividing the inner space into the plurality of areas;
the separation membrane is provided to change a size of a movement passage between adjacent areas; and
the controller controls an operation of the separation membrane based on environment information of each of the plurality of areas.
